(19) United States
(12) Patent Application Publication
(10) Pub. No.: US 2008/0248506 A1
(43) Pub. Date: Oct. 9, 2008

(54) Method of Monitoring Anti-Tumor Activity of an Hdac Inhibitor

(75) Inventors: Kathryn E. Bass, San Francisco, CA (US); Samuel E. Broder, Bethesda, MD (US); Peter R. Young, San Francisco, CA (US)

Correspondence Address: WILSON SONSINI GOODRICH & ROSATI, 650 PAGE MILL ROAD, PALO ALTO, CA, 94304-1050, US

(73) Assignee: Pharmacyclics, Inc., Sunnyvale, CA (US)

(21) Appl. No.: 11/664,885

(22) PCT Filed: Oct. 7, 2005
(86) PCT No.: PCT/US2005/036025

§ 371 Date: Feb. 8, 2008

Related U.S. Application Data
(60) Provisional application No. 60/616,724, filed on Oct. 7, 2004.

(51) Int. Cl. G01N 33/53; G01N 33/68; C12Q 1/02

(52) U.S. Cl. 435/7.92; 436/86; 435/29

(57) ABSTRACT

The present invention relates to the method of determining the anti-tumor activity of a histone deacetylase inhibitor by measuring the phosphorylation of the histone variant H2AX or the level of cytokeratin-18 fragment aa 387-397.

### CROSS-REFERENCE

The Applicants claim priority under 35 U.S.C. 119(e) to copending Provisional Application No. 60/616,724 filed on Oct. 7, 2004, the disclosure of which is incorporated herein by reference in its entirety.

### BACKGROUND OF THE INVENTION

1. Field of Invention

The present invention relates to a method of determining the anti-tumor activity of an histone deacetylase inhibitor by measuring the phosphorylation of the histone variant H2AX or the level of cytokeratin-18 fragment (aa fragment 387-397) formed by cleavage of cytokeratin-18 by caspases 3, 6, 7, and/or 9 at residue Asp396.

2. State of the Art

One of the earliest events at the site of DNA double stranded breaks (DSBs) is the phosphorylation of the histone variant H2AX on serine 139, creating a phosphorylated version known as “γ-H2AX”. Treatment of mammalian cells with ionizing radiation leads to DSBs accompanied by the appearance of γ-H2AX within five minutes of treatment. The presence of γ-H2AX is thought to provide a platform for other damage proteins such as 53BP-1, Mre11, Brca1, NBS, or MDC-1 to localize to the break site. γ-H2AX can be visualized as foci by immunofluorescence using specific antibodies. γ-H2AX foci colocalize with other repair proteins including Rad51, NBS1, 53 BP-1, MDC1, HDAC4, and BRCA1. γ-H2AX is thought to play a critical role in retention of these repair factors at the sites of DSBs. It is now widely accepted that the kinase ATM (ataxia telangiectasia mutated protein) is one enzyme that phosphorylates H2AX in response to DSBs (Burma, A, et al. J. Biol. Chem. 2001, 276 (45), 42462-42467), and ATM might function redundantly with the kinase DNA-PK in vivo (Stiff, T. et al. Cancer Research 2004, 64, 2390-2396). It is now mainly concluded that under most normal growth conditions, irradiation induced H2AX phosphorylation is carried out by ATM and DNA-PK in a redundant, overlapping manner. Because DNA DSBs are often lethal, and are produced as a result of several known chemotherapeutic agents, it has been suggested that H2AX phosphorylation may be a useful surrogate indicator of cell killing by such agents (Banath, J. P., and Olive, P. L. Cancer Research 2003, 63, 4347-4350).

Histone deacetylase (HDAC) enzymes are important modifiers of chromatin structure and are known to play a central role in transcriptional regulation. Recent studies have also linked HDAC enzymes with the process of DNA repair (Fernandez-Capetillo, O., and Nussenzweig, A. Proc. Natl. Acad. Sci. USA. 2004, 101(6), 1427-8). It is known that inhibitors of HDAC enzymes can affect the susceptibility of cells to DNA-damaging agents, possibly by inducing histone hyperacetylation and relaxation of chromatin (Kim M. S. et al. Cancer Res. 2003, 63(21), 7291-7300). Some HDAC inhibitors have been shown to enhance DNA damage induced by radiation. MS-275 has been shown to increase the number of irradiated cells that express γ-H2AX foci, but MS-275 by itself does not produce γ-H2AX accumulation (Camphausen, K., et al. Cancer Research 2004, 64, 316-321). Similarly the HDAC inhibitor FK-228 (depsipeptide) has been shown to augment radiation induced cell death, but has not been shown to induce accumulation of γ-H2AX foci (Zhang, Y., et al. Int. J. Cancer 2004, 110(2), 301-308). Trichostatin, an HDAC inhibitor, on the other hand has been reported to cause H2AX phosphorylation in myeloid leukemia cell lines (HL60, K562, MDS92L; n=3).

Since H2AX phosphorylation is a useful surrogate indicator of cell killing by HDAC, there is a need to determine which HDAC inhibitors upon administration, cause H2AX phosphorylation. This information can be used in tailoring the amount of HDAC inhibitor to be administered to a cancer patient to achieve maximal therapeutic effect with minimal amount of the HDAC inhibitor. The present invention fulfills this and related needs.

Cytokeratins are cytoskeletal proteins known as intermediate filaments and they are expressed by epithelial cells. Cytokeratin-18, a type I intermediate filament protein, is found in epithelial cells exclusively (including epithelial-derived tumors) and not in fibroblasts, lymphocytes and other non-epithelial cells. Cytokeratin-18 is cleaved by caspases 3, 6, 7, and/or 9 during apoptosis. Cleaved cytokeratin-18 fragments can be detected in vitro in cell lysates and conditioned media and in vivo in serum and plasma (Biven et al. Apoptosis 8:262-268, (2003) and cell/tumor lysates. Importantly, intact (nondegraded) cytokeratin molecules have not been demonstrated in the circulation. The half-life of cytokeratin fragments in the circulation, depending on the size of the fragment, is 10-15 h (Barak et al., Clin. Biochem. 37:529-540, (2004). Since formation of cytokeratin-18 fragment (aa fragment 387-397) is a useful surrogate indicator of apoptosis, it can be used to determine which HDAC inhibitors upon administration, cause apoptosis. This information can be used in tailoring the amount of HDAC inhibitor to be administered to a cancer patient to achieve maximal therapeutic effect with the minimal amount of the HDAC inhibitor. The present invention fulfills this and related needs.

### SUMMARY OF THE INVENTION

Applicants have observed that treating tumor cells with an HDAC inhibitor leads to the accumulation of γ-H2AX foci as assayed by immunofluorescence microscopy and by Western blotting using an antibody specific for γ-H2AX. γ-H2AX accumulation can be seen following treatment of cells with the HDAC inhibitor. In cells treated with an HDAC inhibitor, Applicants have found that accumulation of γ-H2AX at timepoints before apoptosis has occurred is an indicator of DNA double stranded breaks (DSBs), as well as an indicator of apoptosis. Additionally, Applicants have found that accumulation of cytokeratin-18 fragment aa 387-397 (as assayed by M30 ELISA) in the lysates coincides with the accumulation of γ-H2AX. Hence, accumulation of γ-H2AX and cytokeratin-18 fragment aa 387-397 can be used as biomarkers to monitor anti-tumor activity of the compounds of the present invention. The HDAC inhibitors described in WO 04/092115 and WO 05/019174 are exemplary of compounds that can be used to practice this invention and are incorporated herein by reference in their entireties.

Accordingly, in a first aspect, this invention provides a method of determining the anti-tumor activity of an HDAC inhibitor comprising measuring the level of phosphorylation of the histone variant H2AX before administration of the HDAC inhibitor and after administration at a timepoint before apoptosis can be detected. Preferably, the measurement is made 0 to 10 hours after administration of the HDAC inhibitor. More preferably, the measurement is made 5 minutes to 8 hours after administration of the HDAC inhibitor. Even more preferably the measurement is made 5 minutes to 6 hours after administration of the HDAC inhibitor. Preferably, the HDAC inhibitor is a compound of

a) Formula (I):

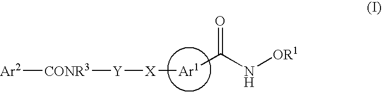

wherein:

R¹ is hydrogen or alkyl;

X is —O—, —NR²—, or —S(O)ₙ where n is 0-2 and R² is hydrogen or alkyl;

Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;

Ar¹ is phenylene or heteroarylene wherein said Ar¹ is optionally substituted with one or two groups independently selected from alkyl, halo, hydroxy, alkoxy, haloalkoxy, or haloalkyl;

R³ is hydrogen, alkyl, hydroxyalkyl, or optionally substituted phenyl; and

Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl; and

individual stereoisomers, individual geometric isomers, or mixtures thereof; or a pharmaceutically acceptable salt thereof; or

b) Formula (II):

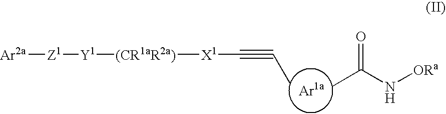

wherein:

R^{a} is hydrogen, alkyl, or alkylcarbonyl;

Ar^{1a} is arylene or heteroarylene wherein said Ar¹ is optionally substituted with one or two substituents independently selected from alkyl, halo, alkoxy, haloalkoxy, or haloalkyl;

X¹ and Y¹ are independently selected from bond or alkylene wherein alkylene is optionally substituted with halo, haloalkyl, hydroxy, alkoxy, haloalkoxy, amino, alkylamino, or dialkylamino;

R^{1a} is hydrogen or alkyl;

R^{2a} is hydrogen, alkyl, halo, haloalkyl, heteroalkyl, substituted heteroalkyl, aryl, heteroaryl, aralkyl, heteroaralkyl, hydroxyalkyl, alkoxyalkyl, or aminoalkyl; or

R^{1a} and R^{2a} together with the carbon to which they are attached form cycloalkylene or heterocycloalkylene;

Z¹ is —CONR^{3a}, —NR⁴CO—, —SO₂NR⁵—, —NR⁶SO₂—, —NR⁷CONR⁸—, —NR⁹SO₂NR¹⁰—, —OCONR¹¹—, or —NR¹²COO— where R³-R¹² are independently selected from hydrogen, alkyl, hydroxyalkyl, haloalkyl, haloalkoxy, alkoxyalkyl, aralkyl, or heteroaralkyl; and

Ar^{2a} is aryl, aralkyl, aralkenyl, heteroaryl, heteroaralkyl, heteroaralkenyl, heterocycloalkyl, or heterocycloalkylalkyl; and

individual stereoisomers, individual geometric isomers, or mixtures thereof; or a pharmaceutically acceptable salt thereof provided that the hydroxamic acid and the acetylenic groups are not ortho to each other. Groups contained in compounds of Formula (I) and (II) are defined in applications WO 04/092115 and WO 05/019174, respectively, and are incorporated herein by reference.

In a second aspect, this invention is directed to a method of determining an efficacious dose for treating a cancer patient of which method comprises administering to the cancer patient different amounts of an HDAC inhibitor and determining, at a timepoint before apoptosis can be detected, the dose of the HDAC inhibitor that causes an increase in phosphorylation of the histone variant H2AX. Preferably, the measurement is made 0 to 12 hours after administration of the HDAC inhibitor. More preferably, the measurement is made 5 minutes to 10 hours after administration of the HDAC inhibitor. Even more preferably the measurement is made 5 minutes to 6 hours after administration of the HDAC inhibitor. Preferably the HDAC inhibitor is a compound of Formula (I) or (II).

In a third aspect, this invention is directed to a method of determining an efficacious dose of an HDAC inhibitor for treating a cancer patient which method comprises:
(i) determining the level of γ-H2AX in the cancer patient prior to the administration of the HDAC inhibitor;
(ii) administering to the cancer patient different amounts of the HDAC inhibitor;
(iii) determining the level of γ-H2AX after administration of the HDAC inhibitor at said different amounts and at a timepoint before apoptosis can be detected; and
(iv) determining the efficacious dose by determining the increase in the level of γH2AX.
   Preferably, the level of γ-H2AX after administration of the HDAC inhibitor is determined 0 to 10 hours after said administration. More preferably, the level of γ-H2AX after administration of the HDAC inhibitor is determined 5 minutes to 8 hours after said administration. Even more preferably, the level of γ-H2AX after administration of the HDAC inhibitor is determined 5 minutes to 6 hours after said administration. Preferably, the HDAC inhibitor is a compound of Formula (I) or Formula (II).

In a fourth aspect, this invention provides a method of determining the anti-tumor activity of a compound of Formula (I):

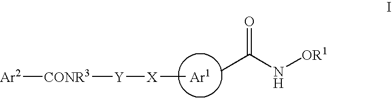

where Ar², R³, Y, X, Ar¹, and R¹ are as defined above; and individual stereoisomers, individual geometric isomers, or mixtures thereof; or a pharmaceutically acceptable salt thereof; which method comprises measuring the level of phosphorylation of the histone variant H2AX and/or level of formation of cytokeratin-18 fragment aa 387-397 before and after administration of the compound of Formula (I).

In a fifth aspect, this invention is directed to a method of determining an efficacious dose of a compound of Formula (I):

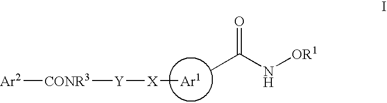

where Ar¹, Ar², R¹, R³, X and Y are as defined above, for treating a cancer patient which method comprises administering to the cancer patient different amounts of a compound of Formula (I) and determining the dose that causes an increase in phosphorylation of the histone variant H2AX and/or formation of cytokeratin-18 fragment aa 387-397 in the patient.

In a sixth aspect, this invention is directed to a method of determining an efficacious dose of a compound of Formula (I):

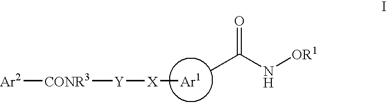

where Ar¹, Ar², R¹, R³, X and Y are as defined above, for treating a cancer patient which method comprises:
(i) determining the level of γ-H2AX in the cancer patient prior to the administration of a compound of Formula (I);
(ii) administering to the cancer patient different amounts of a compound of Formula (I);
(iii) determining the level of γ-H2AX after administration of the compound of Formula (I) at said different amounts; and
(iv) determining the efficacious dose of the compound of Formula (I) by determining the increase in the level of γ-H2AX.

In a seventh aspect, this invention is directed to a method of determining an efficacious dose of a compound of Formula (I):

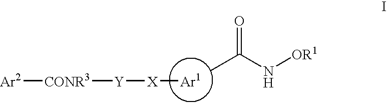

where Ar¹, Ar², R¹, R³, X and Y are as defined above, for treating a cancer patient which method comprises:
(i) determining the level of cytokeratin-18 fragment aa 387-397 in the cancer patient prior to the administration of a compound of Formula (I);
(ii) administering to the cancer patient different amounts of a compound of Formula (I);
(iii) determining the level of cytokeratin-18 fragment aa 387-397 after administration of the compound of Formula (I) at said different amounts; and
(iv) determining the efficacious dose of the compound of Formula (I) by determining the increase in the level of cytokeratin-18 fragment aa 387-397.

The phosphorylation of the histone variant H2AX can be determined utilizing an in vitro assay using blood or cancer tissue samples from the patient. Preferably, the level of the phosphorylated histone variant H2AX is determined using an anti-γH2AX antibody. Preferably, the anti-γH2AX antibody specifically binds to the C-terminal phosphorylated serine in the γH2AX histone protein. The level of phosphorylated histone variant H2AX can be detected using ELISA, immunohistochemistry, immunoblotting or flow cytometry.

The level of cytokeratin-18 fragment aa 387-397 can be determined utilizing an in vitro assay using serum or tumor tissue from the patient and evaluating it with an anti-cytokeratin-18 fragment aa 387-397 antibody. Preferably, the antibody is a monoclonal antibody called M30 which recognizes a neoepitope of cytokeratin-18 in the C-terminal domain exposed after the caspase cleavage at residue Asp396 (aa fragment 387-396). The level of cytokeratin-18 fragment aa 387-397 can be detected using ELISA, immunohistochemistry, immunoblotting or flow cytometry. The desired efficacious dose for a compound of Formula (I) or (II) is one that produces the greatest level of antibody-γH2AX complexes or M30-cytokeratin-18 fragment aa 387-397 complexes.

### DETAILED DESCRIPTION OF THE INVENTION

### Definitions

Unless otherwise stated, the following terms used in the specification and claims are defined for the purposes of this Application and have the following meaning:

“Anti-γH2AX antibody” refers to a protein or an antigenically-reactive fragment thereof comprising one or more polypeptides selected from immunoglobulin light chains, immunoglobulin heavy chains, and antigen-binding fragments thereof, which are capable of binding to γH2AX. The antibody includes intact monoclonal and polyclonal immunoglobulins. The “antigenically-reactive fragment” of an anti-γH2AX antibody includes segments of immunoglobulins that retain the ability to bind selectively γH2AX. The antibody or fragment thereof may be a single-chain antibody. The antibody or fragment thereof may be a heavy chain monomer, dimer or trimer, a light chain monomer, dimer or trimer, a dimer consisting of one heavy and one light chain, and the like. One of ordinary skill in the art will appreciate that the isolated or purified antibody or antigenically-reactive fragment thereof include various deletions, additions or substitutions which either do not affect the binding affinity of the antibody or, preferably, enhances the affinity of the antibody for γH2AX. Alterations may also include truncation of non-essential regions of the antibody, such as those not responsible for antigen binding or structure of the antibody.

“Anti-cytokeratin-18 fragment antibody” refers to a protein or an antigenically-reactive fragment thereof comprising one or more polypeptides selected from immunoglobulin light chains, immunoglobulin heavy chains, and antigen-binding fragments thereof, which are capable of binding to cytokeratin-18 fragment aa 387-397. The antibody includes intact monoclonal and polyclonal immunoglobulins. The “antigenically-reactive fragment” of an anti-cytokeratin-18 fragment aa 387-397 antibody includes segments of immunoglobulins that retain the ability to bind selectively cytokeratin-18 fragment aa 387-397. The antibody or fragment thereof may be a single-chain antibody. The antibody or fragment thereof may be a heavy chain monomer, dimer or trimer, a light chain monomer, dimer or trimer, a dimer consisting of one heavy and one light chain, and the like. One of ordinary skill in the art will appreciate that the isolated or purified antibody or antigenically-reactive fragment thereof include various deletions, additions or substitutions which either do not affect the binding affinity of the antibody or, preferably, enhances the affinity of the antibody for cytokeratin-18 fragment aa 387-397. Alterations may also include truncation of non-essential regions of the antibody, such as those not responsible for antigen binding or structure of the antibody.

“C-terminal phosphorylated serine” refers to a phosphorylated serine located within about 25 amino acids of the C-terminus of the H2AX protein. Preferably, the phosphorylated serine is within about 10 amino acids of the C-terminus of the protein, more preferably within about 4 amino acids from the C-terminus of the protein.

The present invention also includes the prodrugs of compounds of Formula (I) and (II). The term prodrug is intended to represent covalently bonded carriers, which are capable of releasing the active ingredient of Formula (I) and (II) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula (I) and (II) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I) and (II)), amides (e.g., trifluoroacetylamino, acetylamino, and the like), and the like. Prodrugs of compounds of Formula (I) and (II) are also within the scope of this invention.

The present invention also includes N-oxide derivatives and protected derivatives of compounds of Formula (I) and (II). For example, when compounds of Formula (I) and (II) contain an oxidizable nitrogen atom, the nitrogen atom can be converted to an N-oxide by methods well known in the art. When compounds of Formula (I) and (II) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula (I) and (II) can be prepared by methods well known in the art.

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) and (II) can exist as tautomers and/or geometric isomers. All possible tautomers and cis and trans isomers, individual and mixtures thereof are within the scope of this invention. Additionally, as used herein the terms alkyl includes all the possible isomeric forms of said alkyl group albeit only a few examples are set forth. Furthermore, when the cyclic groups such as aryl, heteroaryl, heterocycloalkyl are substituted, they include all the positional isomers albeit only a few examples are set forth. Furthermore, all polymorphic forms and hydrates of a compound of Formula (I) and (II) are within the scope of this invention.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Efficacious dose” means the dose of an HDAC inhibitor, preferably a compound of Formula (I) or (II), which:

(1) prevents the disease, i.e., causes the clinical symptoms of the disease not to develop in a mammal that may be exposed to or predisposed to the disease but does not yet experience or display symptoms of the disease;

(2) inhibits the disease, i.e., arresting or reducing the development of the disease or its clinical symptoms; or

(3) relieves the disease, i.e., causing regression of the disease or its clinical symptoms.

Representative compounds of Formula (I) are disclosed in Table I-IV below.

Compounds of Formula (I) where R¹ and R³ are hydrogen, Ar¹ is phenyl and Ar² and Y are as defined in Table I below are:

TABLE I

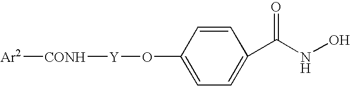

| Cpd | | |
|---|---|---|
| # | Ar² | Y |
| 1 | phenyl | —CH₂—CH₂— |
| 2 | trans phenyl-CH═CH— | —CH₂—CH₂— |
| 3 | trans phenylcyclopropyl | —CH₂—CH₂— |
| 4 | trans 4-MeO-phenyl-CH═CH— | —CH₂—CH₂— |
| 5 | 2-phenylethyl | —CH₂—CH₂— |
| 6 | 1H-indol-3-ylmethyl | —CH₂—CH₂— |
| 7 | thiophen-2-yl | —CH₂—CH₂— |
| 8 | pyridin-3-yl | —CH₂—CH₂— |
| 9 | 4-biphenyl | —CH₂—CH₂— |
| 10 | 3-biphenyl | —CH₂—CH₂— |
| 11 | 5-phenylthiophen-2-yl | —CH₂—CH₂— |
| 12 | thiophen-2-ylmethyl | —CH₂—CH₂— |
| 13 | naphth-2-yl | —CH₂—CH₂— |
| 14 | quinolin-6-yl | —CH₂—CH₂— |
| 15 | 4-phenylthiazol-2-yl | —CH₂—CH₂— |
| 16 | 4-tert-butylphenyl | —CH₂—CH₂— |
| 17 | trans pyridin-3-yl-CH═CH— | —CH₂—CH₂— |
| 18 | 4-pyrrol-1-ylphenyl | —CH₂—CH₂— |
| 19 | 4-(cyclohexene-3-oxy)-phenyl | —CH₂—CH₂— |
| 20 | benzothiazol-2-yl | —CH₂—CH₂— |
| 21 | benzoxazol-2-yl | —CH₂—CH₂— |
| 22 | octahydroisoquinolin-2-ylmethyl | —CH₂—CH₂— |
| 23 | 4-pyridin-4-yl-piperazin-1-ylmethyl | —CH₂—CH₂— |
| 24 | furan-2-yl | —CH₂—CH₂— |
| 25 | 4-(pyridin-3-yl)-phenyl | —CH₂—CH₂— |
| 26 | 4-(pyridin-2-yl)-phenyl | —CH₂—CH₂— |
| 27 | 1H-benzimidazol-2-yl | —CH₂—CH₂— |
| 28 | 1H-pyrrol-2-yl | —CH₂—CH₂— |
| 29 | 4-(benzoylamino)-phenyl | —CH₂—CH₂— |
| 30 | 4-(pyridin-4-yl)-thiazol-2-yl | —CH₂—CH₂— |
| 31 | adamantan-1-yl | —CH₂—CH₂— |
| 32 | 2,4-difluorophenyl | —CH₂—CH₂— |
| 33 | trans 3,4-methylenedioxyphenylCH═CH— | —CH₂—CH₂— |
| 34 | 3,4-methylenedioxyphenyl | —CH₂—CH₂— |
| 35 | 3,4-dimethoxyphenyl | —CH₂—CH₂— |
| 36 | 3,5-dimethoxyphenyl | —CH₂—CH₂— |
| 37 | 3,4-difluorophenyl | —CH₂—CH₂— |
| 38 | 2,5-dimethylphenyl | —CH₂—CH₂— |
| 39 | 2,3-dichlorophenyl | —CH₂—CH₂— |
| 40 | 2,3-dimethylphenyl | —CH₂—CH₂— |
| 41 | 4-chloro-2-methoxyphenyl | —CH₂—CH₂— |
| 42 | 3-ethoxyphenyl | —CH₂—CH₂— |
| 43 | 4-methoxy-2-methylphenyl | —CH₂—CH₂— |
| 44 | 3-fluoro-4-methoxyphenyl | —CH₂—CH₂— |
| 45 | 2-(thiophen-2-ylmethoxy)phenyl | —CH₂—CH₂— |
| 46 | 3-(thiophen-2-ylmethoxy)-phenyl | —CH₂—CH₂— |
| 47 | 2-phenylphenyl | —CH₂—CH₂— |
| 48 | 1H-indol-5-yl | —CH₂—CH₂— |
| 49 | 1H-indol-3-yl | —CH₂—CH₂— |
| 50 | quinolin-3-yl | —CH₂—CH₂— |
| 51 | quinolin-8-yl | —CH₂—CH₂— |
| 52 | 1H-indazol-3-yl | —CH₂—CH₂— |
| 53 | 1H-benzotriazol-5-yl | —CH₂—CH₂— |
| 54 | isoquinolin-1-yl | —CH₂—CH₂— |
| 55 | isoquinolin-3-yl | —CH₂—CH₂— |
| 56 | quinoxalin-2-yl | —CH₂—CH₂— |
| 57 | naphth-1-yl | —CH₂—CH₂— |
| 58 | quinolin-2-yl | —CH₂—CH₂— |
| 59 | 2-pyrrol-1-yl-phenyl | —CH₂—CH₂— |
| 60 | 4-fluoronaphth-1-yl | —CH₂—CH₂— |
| 61 | 1H-benzimidazol-5-yl | —CH₂—CH₂— |
| 62 | 1-methyl-indol-3-yl | —CH₂—CH₂— |
| 63 | 4-MeO-quinolin-2-yl | —CH₂—CH₂— |
| 64 | 3-MeO-naphth-2-yl | —CH₂—CH₂— |
| 65 | 2-MeO-naphth-1-yl | —CH₂—CH₂— |
| 66 | quinolin-4-yl | —CH₂—CH₂— |
| 67 | trans phenyl-CH═C(CH₃)— | —CH₂—CH₂— |
| 68 | 2-N,N-dimethylaminomethylbenzofuran-5-yl | —CH₂—CH₂— |
| 69 | indolin-1-yl | —CH₂—CH₂— |
| 70 | 1,2,3,4-tetrahydroquinolin-1-yl | —CH₂—CH₂— |
| 71 | trans 5-hydroxybenzofuran-2-yl —C(CH₃)═CH— | -(S)-CH(CH₂CH₃)—CH₂— |
| 72 | trans 5-(1-cyclopropylpiperidin-4-yloxy)benzofuran- | -(S)-CH(CH₂CH₃)—CH₂— |
| | 2-yl —C(CH₃)═CH— | |
| 73 | benzofuran-2-yl | -(S)-CH(2-phenylethyl)-CH₂— |
| 74 | 5-(1-cyclopropylpiperidin-4-yloxy)benzofuran-2-yl | —CH₂—CH₂— |
| 75 | 5-(1-cyclopropylpiperidin-4-yloxy)benzofuran-2-yl | -(S)-CH(CH₂CH₃)—CH₂— |
| 76 | 5-(1-cyclopropylpiperidin-4-yloxy)benzofuran-2-yl | -(R)-CH₂—CH(CH₃)— |
| 77 | 5-[1-(2,2,2-trifluoroethyl)piperidin-4- | —CH₂—CH₂— |
| | yloxy]benzofuran-2-yl | |
| 78 | benzofuran-2-yl | -(R)-CH(benzylSO₂- |
| | | methyl)-CH₂— |
| 79 | benzofuran-2-yl | —(R)—CH(benzylS-methyl)- |
| | | CH₂— |
| 80 | trans 5-methoxybenzofuran-2-yl —C(CH₃)═CH— | —CH₂—CH₂— |
| 81 | 1,2,3,4-tetrahydroisoquinolin-2-yl | —CH₂—CH₂— |
| 82 | isoindolin-2-yl | —CH₂—CH₂— |
| 83 | morpholin-4-yl | —CH₂—CH₂— |
| 84 | 4-benzyl-piperazin-1-yl | —CH₂—CH₂- |
| 85 | (R)-3-HO-pyrrolidin-1-yl | —CH₂—CH₂— |
| 86 | piperidin-1-yl | —CH₂—CH₂— |
| 87 | 6-CH₃-1,2,3,4-tetrahydroquinolin-1-yl | —CH₂—CH₂— |
| 88 | 2-CH₃-indolin-1-yl | —CH₂—CH₂— |
| 89 | 6-F-2-CH₃-1,2,3,4-tetrahydroquinolin-1-yl | —CH₂—CH₂— |
| 90 | isoindolin-1-yl | -(S)-CH(CH₂CH₃)—CH₂— |
| 91 | trans phenyl-CH═CH— | —CH₂—CH₂—CH₂— |
| 92 | trans 4-CH₃O-phenyl-CH═CH— | —CH₂—CH₂—CH₂— |
| 93 | 4-phenylthiazol-2-yl | —CH₂—CH₂—CH₂— |
| 94 | trans phenyl-CH═CH— | -(S)-CH(methyl)-CH₂— |
| 95 | trans phenyl-CH═CH— | -(R)-CH(methyl)-CH₂— |
| 96 | trans phenyl-CH═CH— | -(S)-CH(i-butyl)-CH₂— |
| 97 | trans phenyl-CH═CH— | -(S)-CH(cyclohexyl- |
| | | methyl)-CH₂— |
| 98 | trans phenyl-CH═CH— | -(S)-CH(i-propyl)-CH₂— |
| 99 | trans phenyl-CH═CH— | -(S)-CH(benzyl)-CH₂— |
| 100 | trans phenyl-CH═CH— | -(R)-CH(benzyl)-CH₂— |
| 101 | trans phenyl-CH═CH— | -(R)-CH(i-butyl)-CH₂— |
| 102 | trans phenyl-CH═CH— | -(R)-CH(i-propyl)-CH₂— |
| 103 | trans phenyl-CH═CH— | -(RS)-CH(n-butyl)-CH₂— |
| 104 | trans phenyl-CH═CH— | -(RS)-CH(4-Cl-benzyl)-CH₂— |
| 105 | trans phenyl-CH═CH— | -(S)-CH(CH₂CH₃)—CH₂— |
| 106 | trans phenyl-CH═CH— | -(R)-CH(CH₂CH₃)—CH₂— |
| 107 | trans phenyl-CH═CH— | -(S)-CH(2-MeS-ethyl)-CH₂— |
| 108 | trans phenyl-CH═CH— | -(R)-CH(2-MeS-ethyl)-CH₂— |
| 109 | trans phenyl-CH═CH— | -(S)-CH(phenyl)-CH₂— |
| 110 | trans phenyl-CH═CH— | -(R)-CH(phenyl)-CH₂— |
| 111 | trans phenyl-CH═CH— | -(S)-CH(2-MeSO₂-ethyl)-CH₂— |
| 112 | trans phenyl-CH═CH— | -(R)-CH(2-MeSO₂-ethyl)-CH₂— |
| 113 | trans phenyl-CH═CH— | -(R)-CH(benzylSO₂- |
| | | methyl)-CH₂— |
| 114 | thiophen-2-yl | -(S)-CH(CH₂CH₃)—CH₂— |
| 115 | 4-biphenyl | -(S)-CH(CH₂CH₃)—CH₂— |
| 116 | naphth-2-yl | -(S)-CH(CH₂CH₃)—CH₂— |
| 117 | trans phenyl-CH═CH— | -(R)-CH(benzyl-S-methyl)- |
| | | CH₂— |
| 118 | phenyl | -(S)-CH(CH₂CH₃)—CH₂— |
| 119 | benzyl | -(S)-CH(CH₂CH₃)—CH₂— |
| 120 | 2-phenylethyl | -(S)-CH(CH₂CH₃)—CH₂— |
| 121 | trans phenyl-CH═CH— | -(S)-CH(hydroxy-methyl)- |
| | | CH₂— |
| 122 | 4-phenylthiazol-2-yl | -(S)-CH(CH₂CH₃)—CH₂— |
| 123 | trans 4-CH₃O-phenyl-CH═CH— | -(S)-CH(CH₂CH₃)—CH₂— |
| 124 | 2-N,N-dimethylaminomethyl-benzofuran-5-yl- | -(S)-CH(CH₂CH₃)—CH₂— |
| 125 | trans phenyl-CH═CH— | -(R)-CH₂—CH(CH₃)— |
| 126 | trans phenyl-CH═CH— | -(S)-CH₂—CH(CH₃)— |
| 127 | 4-phenyl-thiazol-2-yl | -(R)-CH₂—CH(CH₃)— |
| 128 | 4-phenyl-thiazol-2-yl | -(S)-CH₂—CH(CH₃)— |
| 129 | 4-biphenyl | -(R)-CH₂—CH(CH₃)— |
| 130 | trans 4-CH₃O-phenyl-CH═CH— | -(R)-CH₂—CH(CH₃)— |
| 131 | 4-(2-pyridin-4-ylthiazol-5-yl)phenyl | —CH₂—CH₂— |
| 132 | 7-chloro-4-methylbenzofuran-2-yl | —CH₂—CH₂— |
| 133 | 4-[2-(4-methylpiperazin-1-yl)thiazol-5-yl]-phenyl | —CH₂—CH₂— |
| 134 | 4-(2-pyridin-4-ylaminothiazol-5-yl)phenyl | —CH₂—CH₂— |
| 135 | 4-(4-methylpiperazin-1-yl)phenyl | —CH₂—CH₂— |
| 136 | 4-(4-hydroxypiperidin-1-yl)phenyl | —CH₂—CH₂— |
| 137 | 4-(4-morpholin-4-ylmethylthiazol-2-yl)phenyl | —CH₂—CH₂— |
| 138 | 7-fluoro-4-methylbenzofuran-2-yl | —CH₂—CH₂— |
| 139 | 7-fluoro-4-(2-methoxyethoxylmethyl)-benzofuran-2-yl | —CH₂—CH₂— |
| 140 | 4-hydroxyquinolin-2-yl | —CH₂—CH₂— |
| 141 | 7-fluoro-4-phenoxymethylbenzofuran-2-yl | —CH₂—CH₂— |
| 143 | 4-[2-(4-methylpiperazin-1-ylmethyl)thiazol-5- | —CH₂—CH₂— |
| | yl]phenyl | |
| 144 | pyridin-2-yl | —CH₂—CH₂— |
| 145 | 3-hydroxypyridin-2-yl | —CH₂—CH₂— |
| 146 | 6-hydroxypyridin-2-yl | —CH₂—CH₂— |
| 147 | 6-(4-nitrophenoxy)pyridin-2-yl | —CH₂—CH₂— |
| 148 | 4-(2-methoxyethoxy)quinolin-2-yl | —CH₂—CH₂— |
| 149 | 4-(2-dimethylaminoethoxy)quinolin-2-yl | —CH₂—CH₂— |
| 150 | 6-bromopyridin-2-yl | —CH₂—CH₂— |
| 151 | 5-bromopyridin-3-yl | —CH₂—CH₂— |
| 152 | 4-methoxyquinolin-2-yl | -(S)-CH(CH₂CH₃)—CH₂— |
| 153 | 1-methoxynaphth-2-yl | —CH₂—CH₂— |
| 154 | 4-methoxyquinolin-2-yl | -(R)-CH₂—CH(CH₃)— |
| 155 | 5-phenylpyridin-3-yl | —CH₂—CH₂— |
| 156 | 6-benzyloxypyridin-2-yl | —CH₂—CH₂— |
| 157 | 6-(2-methylpropyloxy)pyridin-2-yl | —CH₂—CH₂— |
| 158 | 6-(2-phenylethyloxy)pyridin-2-yl | —CH₂—CH₂— |
| 159 | 4-(3,3,3-trifluoropropyloxy)quinolin-2-yl | —CH₂—CH₂— |
| 160 | 4-(3,3,3-trifluoropropyloxy)quinolin-2-yl | -(S)-CH(CH₂CH₃)—CH₂— |
| 161 | 4-(3,3,3-trifluoropropyloxy)quinolin-2-yl | -(R)-CH₂—CH(CH₃)— |
| 162 | trans 3-hydroxyphenyl-CH═CH— | —CH₂—CH₂— |
| 163 | trans 4-hydroxyphenyl-CH═CH— | —CH₂—CH₂— |
| 164 | 3′-(2-hydroxyethyl)biphen-4-yl | —CH₂—CH₂— |
| 165 | 3′-(2-hydroxyethyl)biphen-3-yl | —CH₂—CH₂— |
| 166 | 2′-(2-hydroxyethyl)biphen-4-yl | —CH₂—CH₂— |
| 167 | trans benzofuran-2-yl-CH═CH— | —CH₂—CH₂— |
| 168 | 2′-(2-hydroxyethyl)biphen-3-yl | —CH₂—CH₂— |
| 169 | 5-thiophen-3-ylpyridin-3-yl | —CH₂—CH₂— |
| 170 | 6-(4-acetylaminophenoxy)pyridin-2-yl | —CH₂—CH₂— |
| 171 | 6-(4-aminophenoxy)pyridin-2-yl | —CH₂—CH₂— |
| 172 | trans 2-methoxyphenyl-CH═CH— | —CH₂—CH₂— |
| 173 | trans 3-methoxyphenyl-CH═CH— | —CH₂—CH₂— |
| 174 | 5-(4-dimethylaminophenyl)pyridin-3-yl | —CH₂—CH₂— |
| 175 | trans 5-bromothiophen-2-yl-CH═CH— | —CH₂—CH₂— |
| 176 | trans furan-3-yl-CH═CH— | —CH₂—CH₂— |
| 177 | trans thiophen-3-yl-CH═CH— | —CH₂—CH₂— |
| 178 | trans thiophen-2-yl-CH═CH— | —CH₂—CH₂— |
| 179 | trans 3-tolyl-CH═CH— | —CH₂—CH₂— |
| 180 | trans 4-tolyl-CH═CH— | —CH₂—CH₂— |
| 181 | trans benzofuran-2-yl-C(CH₃)═CH— | —CH₂—CH₂— |
| 182 | cis benzofuran-2-yl-C(CH₃)═CH— | —CH₂—CH₂— |
| 183 | trans 4-dimethylaminophenyl-CH═CH— | —CH₂—CH₂— |
| 184 | trans indol-3-yl-CH═CH— | —CH₂—CH₂— |
| 185 | trans 2-tolyl-CH═CH— | —CH₂—CH₂— |
| 186 | trans 2-hydroxyphenyl-CH═CH— | —CH₂—CH₂— |
| 187 | trans 7-methoxybenzofuran-2-yl-CH═CH— | —CH₂—CH₂— |
| 188 | trans 7-methoxybenzofuran-2-yl-CH═CH— | -(R)-CH(CH₂CH₃)—CH₂— |
| 189 | trans 5-methoxybenzofuran-2-yl-C(CH₃)═CH— | -(S)-CH(CH₂CH₃)—CH₂— |
| 190 | trans furan-2-yl-CH═CH— | —CH₂—CH₂— |
| 191 | 4-[4-(2-morpholin-4-ylethyl)thiazol-2-yl]phenyl | —CH₂—CH₂— |

and are named as:
N-hydroxy-4-(2-benzenecarbonylamino-ethoxy)benzamide;
N-hydroxy-4-(2-trans-cinnamoylaminoethoxy)benzamide;
N-hydroxy-4-(2-trans-2-phenylcyclopropylcarbonylaminoethoxy)benzamide;
N-hydroxy-4-(2-trans-4-methoxycinnamoylaminoethoxy)benzamide;
N-hydroxy-4-[2-(2-phenylethylcarbonylamino)ethoxy]benzamide;
N-hydroxy-4-[2-(1H-indol-3-ylmethylcarbonylamino)ethoxy]benzamide;
N-hydroxy-4-(2-thiophen-2-ylcarbonylaminoethoxy)benzamide;
N-hydroxy-4-(2-pyridin-3-ylcarbonylaminoethoxy)benzamide;
N-hydroxy-4-(2-biphen-4-ylcarbonylaminoethoxy)benzamide;
N-hydroxy-4-(2-biphen-3-ylcarbonylaminoethoxy)benzamide;
N-hydroxy-4-[2-(5-phenylthiophen-2-ylcarbonylamino)ethoxy]benzamide;
N-hydroxy-4-[2-(thiophen-2-ylmethylcarbonylamino)ethoxy]benzamide;
N-hydroxy-4-[2-(napth-2-ylcarbonylamino)ethoxy]benzamide;
N-hydroxy-4-[2-(quinolin-6-ylcarbonylamino)ethoxy]benzamide;
N-hydroxy-4-[2-(4-phenylthiazol-2-ylcarbonylamino)ethoxy]benzamide;
N-hydroxy-4-[2-(4-tert-butylphenylcarbonylamino)ethoxy]benzamide;
N-hydroxy-4-[2-(trans-3-pyridin-3-ylacryloylamino)ethoxy]benzamide;
N-hydroxy-4-[2-(4-pyrrol-1-ylphenylcarbonylamino)ethoxy]benzamide;
N-hydroxy-4-[2-(4-cyclohexene-3-oxyphenylcarbonylamino)ethoxy]benzamide;
N-hydroxy-4-[2-(benzthiazol-2-ylcarbonylamino)ethoxy]benzamide;
N-hydroxy-4-[2-(benzoxazol-2-ylcarbonylamino)ethoxy]benzamide;
N-hydroxy-4-[2-(octahydroisoquinolin-2-ylmethylcarbonylamino)ethoxy]benzamide;
N-hydroxy-4-[2-(4-pyridin-4-ylpiperazin-1-ylmethylcarbonylaminio)ethoxy]benzamide;
N-hydroxy-4-[2-(furan-2-ylcarbonylamino)ethoxy]benzamide;
N-hydroxy-4-[2-(4-pyridin-3-ylphenylcarbonylamino)ethoxy]benzamide;
N-hydroxy-4-[2-(4-pyridin-2-ylphenylcarbonylamino)ethoxy]benzamide;
N-hydroxy-4-[2-(benzimidazol-2-ylcarbonylamino)ethoxy]benzamide;
N-hydroxy-4-[2-(1H-pyrrol-2-ylcarbonylamino)ethoxy]benzamide;
N-hydroxy-4-[2-(4-benzoylaminophenylcarbonylamino)ethoxy]benzamide;
N-hydroxy-4-[2-(4-pyridin-4-ylthiazol-2-ylcarbonylamino)ethoxy]benzamide;
N-hydroxy-4-[2-(admantan-1-ylcarbonylamino)ethoxy]benzamide;
N-hydroxy-4-[2-(2,4-difluorophenylcarbonylamino)ethoxy]benzamide;
N-hydroxy-4-[2-(3-trans-3,4-methylenedioxyphenylacryloylamino)ethoxy]-benzamide;
N-hydroxy-4-[2-(3,4-methylenedioxyphenylcarbonylamino)ethoxy]benzamide;
N-hydroxy-4-[2-(3,4-dimethoxyphenylcarbonylamino)ethoxy]benzamide;
N-hydroxy-4-[2-(3,5-dimethoxyphenylcarbonylamino)ethoxy]benzamide;
N-hydroxy-4-[2-(3,4-difluorophenylcarbonylamino)ethoxy]benzamide;
N-hydroxy-4-[2-(2,5-dimethylphenylcarbonylamino)ethoxy]benzamide;
N-hydroxy-4-[2-(2,3-dichlorophenylcarbonylamino)ethoxy]benzamide;
N-hydroxy-4-[2-(2,3-dimethylphenylcarbonylamino)ethoxy]benzamide;
N-hydroxy-4-[2-(4-chloro-2-methoxyphenylcarbonylamino)ethoxy]benzamide;
N-hydroxy-4-[2-(3-ethoxyphenylcarbonylamino)ethoxy]benzamide;
N-hydroxy-4-[2-(4-methoxy-2-methylphenylcarbonylamino)ethoxy]benzamide;
N-hydroxy-4-[2-(3-fluoro-4-methoxyphenylcarbonylamino)ethoxy]benzamide;
N-hydroxy-4-[2-(2-thiophen-2-ylmethoxyphenylcarbonylamino)ethoxy]benzamide;
N-hydroxy-4-[2-(3-thiophen-2-ylmethoxyphenylcarbonylamino)ethoxy]benzamide;
N-hydroxy-4-[2-(biphen-2-ylcarbonylamino)ethoxy]benzamide;
N-hydroxy-4-[2-(1H-indol-5-ylcarbonylamino)ethoxy]benzamide;
N-hydroxy-4-[2-(1H-indol-3-ylcarbonylamino)ethoxy]benzamide;
N-hydroxy-4-[2-(quinolin-3-ylcarbonylamino)ethoxy]benzamide;
N-hydroxy-4-[2-(quinolin-8-ylcarbonylamino)ethoxy]benzamide;
N-hydroxy-4-[2-(1H-indazol-3-ylcarbonylamino)ethoxy]benzamide;
N-hydroxy-4-[2-(1H-benzotriazol-5-ylcarbonylamino)ethoxy]benzamide;
N-hydroxy-4-[2-(isoquinolin-1-ylcarbonylamino)ethoxy]benzamide;
N-hydroxy-4-[2-(isoquinolin-3-ylcarbonylamino)ethoxy]benzamide;
N-hydroxy-4-[2-(quinoxalin-2-ylcarbonylamino)ethoxy]benzamide;
N-hydroxy-4-[2-(naphth-1-ylcarbonylamino)ethoxy]benzamide;
N-hydroxy-4-[2-(quinolin-2-ylcarbonylamino)ethoxy]benzamide;
N-hydroxy-4-[2-(2-pyrrol-1-ylphenylcarbonylamino)ethoxy]benzamide;
N-hydroxy-4-[2-(4-fluoronapth-1-ylcarbonylamino)ethoxy]benzamide;
N-hydroxy-4-[2-(1H-benzimidazol-5-ylcarbonylamino)ethoxy]benzamide;
N-hydroxy-4-[2-(1-methylindol-3-ylcarbonylamino)ethoxy]benzamide;
N-hydroxy-4-[2-(4-methoxyquinolin-2-ylcarbonylamino)ethoxy]benzamide;
N-hydroxy-4-[2-(3-methoxynapth-2-ylcarbonylamino)ethoxy]benzamide;
N-hydroxy-4-[2-(2-methoxynapth-1-ylcarbonylamino)ethoxy]benzamide;
N-hydroxy-4-[2-(quinolin-4-ylcarbonylamino)ethoxy]benzamide;
N-hydroxy-4-[2-(trans-2-methylcinnamoylamino)ethoxy]benzamide;
N-hydroxy-4-[2-(2-N,N-dimethylaminomethylbenzofuran-5-ylcarbonylamino)ethoxy]-benzamide;
N-hydroxy-4-(2-indolin-1-ylcarbonylaminoethoxy)benzamide;
N-hydroxy-4-[2-(1,2,3,4-tetrahydroquinolin-1-ylcarbonylamino)ethoxy]benzamide;
N-hydroxy-4-{2S-[trans-3-(5-hydroxybenzofuran-2-yl)but-2-enoylamino]butoxy}benzamide;
N-hydroxy-4-{2S-[trans-3-(5-(1-cyclopropylpiperidin-4-yloxy)benzofuran-2-yl)but-2-enoyl-amino]butoxy}benzamide;
N-hydroxy-4-[2S-(benzofuran-2-ylcarbonylamino)-4-phenylbutoxy)benzamide;
N-hydroxy-4-{2-[5-(1-cyclopropylpiperidin-4-yloxy)benzofuran-2-ylcarbonylamino]-ethoxy}benzamide;
N-hydroxy-4-{2S-[5-(1-cyclopropylpiperidin-4-yloxy)benzofuran-2-ylcarbonylamino]-butoxy}benzamide;
N-hydroxy-4-{2-[5-(1-cyclopropylpiperidin-4-yloxy)benzofuran-2-ylcarbonylamino]-1R-methylethoxy}benzamide;
N-hydroxy-4-{2-[5-(1-(2,2,2-trifluoroethyl)piperidin-4-yloxy)benzofuran-2-ylcarbonyl-amino]-ethoxy}benzamide;
N-hydroxy-4-[2R-(benzofuran-2-ylcarbonylamino)-3-benzylsulfonylpropoxy]benzamide;
N-hydroxy-4-[2R-(benzofuran-2-ylcarbonylamino)-3-benzylthiopropoxy]benzamide;
N-hydroxy-4-[2-(trans-3-(5-methoxybenzofuran-2-yl)but-2-enoylcarbonylamino)-ethoxy]benzamide;
N-hydroxy-4-[2-(1,2,3,4-tetrahydroisoquinolin-2-ylcarbonylamino)ethoxy]benzamide;
N-hydroxy-4-[2-(isoindolin-2-ylcarbonylamino)ethoxy]benzamide;
N-hydroxy-4-[2-(morpholin-4-ylcarbonylamino)ethoxy]benzamide;
N-hydroxy-4-[2-(4-benzylpiperazin-1-ylcarbonylamino)ethoxy]benzamide;
N-hydroxy-4-[2-(3(R)-hydroxypyrrolidin-1-ylcarbonylamino)ethoxy]benzamide;
N-hydroxy-4-[2-(piperidin-1-ylcarbonylamino)ethoxy]benzamide;
N-hydroxy-4-[2-(6-methyl-1,2,3,4-tetrahydroquinolin-1-ylcarbonylamino)ethoxy]-benzamide;
N-hydroxy-4-[2-(2-methylindolin-1-ylcarbonylamino)ethoxy]benzamide;
N-hydroxy-4-[2-(6-fluoro-2-methyl-1,2,3,4-tetrahydroquinolin-1-ylcarbonylamino)-ethoxy]-benzamide;
N-hydroxy-4-[2S-(isoindolin-1-ylcarbonylamino)butoxy]benzamide;
N-hydroxy-4-[3-(trans-cinnamoylamino)propoxy]benzamide;
N-hydroxy-4-[3-(trans-4-methoxycinnamoylamino)propoxy]benzamide;
N-hydroxy-4-[3-(4-phenylthiazol-2-ylcarbonylamino)propoxy]benzamide;
N-hydroxy-4-[2S-(trans-cinnamoylamino)propoxy)benzamide;
N-hydroxy-4-[2R-(trans-cinnamoylamino)propoxy)benzamide;
N-hydroxy-4-[2S-(trans-cinnamoylamino)-4-methylpentoxy]benzamide;
N-hydroxy-4-[2S-(trans-cinnamoylamino)-3-cyclohexylpropoxy]benzamide;
N-hydroxy-4-[2S-(trans-cinnamoylamino)-3-methylbutoxy]benzamide;
N-hydroxy-4-[2S-(trans-cinnamoylamino)-3-phenylpropoxy]benzamide;
N-hydroxy-4-[2R-(trans-cinnamoylamino)-3-phenylpropoxy]benzamide;
N-hydroxy-4-[2R-(trans-cinnamoylamino)-4-methylpentoxy]benzamide;
N-hydroxy-4-[2R-(trans-cinnamoylamino)-3-methylbutoxy]benzamide;
N-hydroxy-4-[2RS-(trans-cinnamoylamino)hexyloxy]benzamide;
N-hydroxy-4-[2RS-(trans-cinnamoylamino)-3-(4-chlorophenyl)propoxy)benzamide;
N-hydroxy-4-[2S-(trans-cinnamoylamino)butoxy]benzamide;
N-hydroxy-4-[2R-(trans-cinnamoylamino)butoxy]benzamide;
N-hydroxy-4-[2S-(trans-cinnamoylamino)-4-methylthiobutoxy)benzamide;
N-hydroxy-4-[2R-(trans-cinnamoylamino)-4-methylthiobutoxy]benzamide;
N-hydroxy-4-[2S-(trans-cinnamoylamino)-2-phenylethoxy]benzamide;
N-hydroxy-4-[2R-(trans-cinnamoylamino)-2-phenylethoxy]benzamide;
N-hydroxy-4-[2S-(trans-cinnamoylamino)-4-methylsulfonylbutoxy]benzamide;
N-hydroxy-4-[2R-(trans-cinnamoylamino)-4-methylsulfonylbutoxy]benzamide;
N-hydroxy-4-[2R-(trans-cinnamoylamino)-3-benzylsulfonylpropoxy]benzamide;
N-hydroxy-4-[2S-(thiophen-2-ylcarbonylamino)butoxy]benzamide;
N-hydroxy-4-[2S-(biphen-4-ylcarbonylamino)butoxy]benzamide;
N-hydroxy-4-[2S-(naphth-2-ylcarbonylamino)butoxy]benzamide;
N-hydroxy-4-[2R-(trans-cinnamoylamino)-3-benzylthiopropoxy]benzamide;
N-hydroxy-4-[2S-(benzenecarbonylamino)butoxy]benzamide;
N-hydroxy-4-[2S-(benzylcarbonylamino)butoxy]benzamide;
N-hydroxy-4-[2S-(2-phenylethylcarbonylamino)butoxy]benzamide;
N-hydroxy-4-[2S-(trans-cinnanoylamino)-3-hydroxypropoxy]benzamide;
N-hydroxy-4-[2S-(4-phenylthiazol-2-ylcarbonylamino)butoxy]benzamide;
N-hydroxy-4-[2S-(trans-4-methoxycinnamoylamino)butoxy]benzamide;
N-hydroxy-4-[2S-(2-N,N-dimethylaminomethylbenzofuran-5-ylcarbonylamino)butoxy]-benzamide;
N-hydroxy-4-[2-(trans-cinnamoylamino)-1R-methylethoxy]benzamide;
N-hydroxy-4-[2-(trans-cinnamoylamino)-1S-methylethoxy)benzamide;
N-hydroxy-4-[2-(4-phenylthiazol-2-ylcarbonylamino)-1R-methylethoxy]benzamide;
N-hydroxy-4-[2-(4-phenylthiazol-2-ylcarbonylamino)-1S-methylethoxy]benzamide;
N-hydroxy-4-[2-(biphen-4-ylcarbonylamino)-1R-methylethoxy]benzamide;
N-hydroxy-4-[2-(trans-4-methoxycinnamoylamino)-1R-methylethoxy]benzamide;
N-hydroxy-4-{2-[4-(2-pyridin-2-ylthiazol-5-yl)phenylcarbonylamino]ethoxy}benzamide;
N-hydroxy-4-[2-(7-chloro-4-methylbenzofuran-2-ylcarbonylamino)ethoxy]benzamide;
N-hydroxy-4-{2-[4-(2-(4-methylpiperazin-1-yl)thiazol-5-yl)phenylcarbonylamino]-ethoxy}-benzamide;
N-hydroxy-4-{2-[4-(2-pyridin-4-ylaminothiazol-5-yl)phenylcarbonylamino]ethoxy}-benzamide;
N-hydroxy-4-{2-[4-(4-methylpiperazin-1-yl)phenylcarbonylamino]ethoxy}benzamide;
N-hydroxy-4-{2-[4-(4-hydroxypiperidin-1-yl)phenylcarbonylamino]ethoxy}benzamide;
N-hydroxy-4-{2-[4-(4-morpholin-4-ylmethylthiazol-5-yl)phenylcarbonylamino]ethoxy}-benzamide;
N-hydroxy-4-[2-(7-fluoro-4-methylbenzofuran-2-ylcarbonylamino)ethoxy]benzamide;
N-hydroxy-4-{2-[7-fluoro-4-(2-methoxyethoxymethyl)benzofuran-2-ylcarbonylamino)-ethoxy}benzamide;
N-hydroxy-4-[2-(4-hydroxyquinolin-2-ylcarbonylamino)ethoxy]benzamide;
N-hydroxy-4-[2-(7-fluoro-4-phenoxymethylbenzofuran-2-ylcarbonylamino)ethoxy]-benzamide;
N-hydroxy-4-{2-[4-(2-(4-methylpiperazin-1-ylmethyl)thiazol-5-yl)phenylcarbonyl-amino]-ethoxy}benzamide;
N-hydroxy-4-[2-(pyridin-2-ylcarbonylamino)ethoxy]benzamide;
N-hydroxy-4-[2-(3-hydroxypyridin-2-ylcarbonylamino)ethoxy]benzamide;
N-hydroxy-4-[2-(6-hydroxypyridin-2-ylcarbonylamino)ethoxy]benzamide;
N-hydroxy-4-{2-[6-(4-nitrophenoxy)pyridin-2-ylcarbonylamino]ethoxy}benzamide;
N-hydroxy-4-{2-[4-(2-methoxyethoxy)quinolin-2-ylcarbonylamino]ethoxy}benzamide;
N-hydroxy-4-{2-[4-(2-N,N-dimethylaminoethoxy)quinolin-2-ylcarbonylamino]ethoxy}-benzamide;
N-hydroxy-4-[2-(6-bromopyridin-2-ylcarbonylamino)ethoxy]benzamide;
N-hydroxy-4-[2-(5-bromopyridin-3-ylcarbonylamino)ethoxy]benzamide;
N-hydroxy-4-[2S-(4-methoxyquinolin-2-ylcarbonylamino)butoxy]benzamide;
N-hydroxy-4-[2-(1-methoxynaphth-2-ylcarbonylamino)ethoxy]benzamide;
N-hydroxy-4-[2-(4-methoxyquinolin-2-ylcarbonylamino)-1R-methylethoxy]benzamide;
N-hydroxy-4-[2-(5-phenylpyridin-3-ylcarbonylamino)ethoxy]benzamide;
N-hydroxy-4-[2-(6-benzyloxypyridin-2-ylcarbonylamino)ethoxy]benzamide;
N-hydroxy-4-{2-[6-(2-methylpropyloxy)pyridin-2-ylcarbonylamino]ethoxy}benzamide;
N-hydroxy-4-{2-[6-(2-phenylethyloxy)pyridin-2-ylcarbonylamino]ethoxy}benzamide;
N-hydroxy-4-{2-[4-(3,3,3-trifluoropropyloxy)quinolin-2-ylcarbonylamino]ethoxy}-benzamide;
N-hydroxy-4-{2S-[4-(3,3,3-trifluoropropyloxy)quinolin-2-ylcarbonylamino]butoxy}-benzamide;
N-hydroxy-4-{2-[4-(3,3,3-trifluoropropyloxy)quinolin-2-ylcarbonylamino]-1R-methyl-ethoxy}benzamide;
N-hydroxy-4-[2-(trans-3-hydroxycinnamoylamino)ethoxy]benzamide;
N-hydroxy-4-[2-(trans-4-hydroxycinnamoylamino)ethoxy]benzamide;
N-hydroxy-4-{2-[3′-(2-hydroxyethyl)biphen-4-ylcarbonylamino]ethoxy}benzamide;
N-hydroxy-4-{2-[3′-(2-hydroxyethyl)biphen-3-ylcarbonylamino]ethoxy}benzamide;
N-hydroxy-4-{2-[2′-(2-hydroxyethyl)biphen-4-ylcarbonylamino]ethoxy}benzamide;
N-hydroxy-4-[2-(trans-2-benzofuran-2-ylacryloylamino)ethoxy]benzamide;
N-hydroxy-4-{2-[2′-(2-hydroxyethyl)biphen-3-ylcarbonylamino]ethoxy}benzamide;
N-hydroxy-4-{2-[5-(thiophen-3-yl)pyridin-3-ylcarbonylamino]ethoxy}benzamide;
N-hydroxy-4-{2-[6-(4-acetylaminophenoxy)pyridin-2-ylcarbonylamino]ethoxy}-benzamide;
N-hydroxy-4-{2-[6-(4-aminophenoxy)pyridin-2-ylcarbonylamino]ethoxy}benzamide;
N-hydroxy-4-[2-(trans-2-methoxycinnamoylamino)ethoxy]benzamide;
N-hydroxy-4-[2-(trans-3-methoxycinnamoylamino)ethoxy]benzamide;
N-hydroxy-4-{2-[5-(4-dimethylaminophenyl)pyridin-3-ylcarbonylamino]ethoxy}-benzamide;
N-hydroxy-4-{2-[trans-3-(5-bromothiophen-2-yl)acryloylamino]ethoxy}benzamide;
N-hydroxy-4-[2-(trans-3-furan-3-ylacryloylamino)ethoxy]benzamide;
N-hydroxy-4-[2-(trans-3-thiophen-3-ylacryloylamino)ethoxy]benzamide;
N-hydroxy-4-[2-(trans-thiophen-2-ylacryloylamino)ethoxy]benzamide;
N-hydroxy-4-{2-[trans-3-methylcinnamoylamino]ethoxy}benzamide;
N-hydroxy-4-{2-[trans-4-methylcinnamoylamino]ethoxy}benzamide;
N-hydroxy-4-{2-[trans-3-(benzofuran-2-yl)but-2-enoylamino]ethoxy}benzamide;
N-hydroxy-4-{2-[cis-3-(benzofuran-2-yl)but-2-enoylamino]ethoxy}benzamide;
N-hydroxy-4-[2-(trans-4-dimethylaminocinnamoylamino)ethoxy]benzamide;
N-hydroxy-4-[2-(trans-3-indol-3-ylacryloylamino)ethoxy]benzamide;
N-hydroxy-4-{2-[trans-2-methylcinnamoylamino]ethoxy}benzamide;
N-hydroxy-4-[2-(trans-2-hydroxycinnamoylamino)ethoxy]benzamide;
N-hydroxy-4-{2-[trans-3-(7-methoxybenzofuran-2-yl)acryloylamino]ethoxy}benzamide;
N-hydroxy-4-{2R-[trans-3-(7-methoxybenzofuran-2-yl)acryloylamino]butoxy}benzamide;
N-hydroxy-4-{2S-[trans-3-(5-methoxybenzofuran-2-yl)but-2-enoylamino]butoxy}benzamide;
N-hydroxy-4-[2-(trans-3-furan-2-ylacryloylamino)ethoxy]benzamide; and
N-hydroxy-4-{2-[4-(4-(2-morpholin-4-ylethyl)thiazol-2-yl)phenylcarbonylamino]ethoxy}-benzamide.

Compounds of Formula (I) where R¹ is hydrogen, Ar¹ is phenyl and R³, Ar² and Y are as defined in Table II below are:

TABLE II

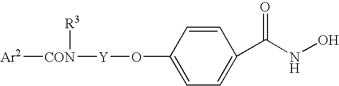

| Cpd | | | |
|---|---|---|---|
| # | R³ | Ar² | Y |
| 1 | 2-HO-ethyl | trans phenyl-CH═CH— | —CH₂—CH₂— |
| 2 | phenyl | trans phenyl-CH═CH— | —CH₂—CH₂— |
| 3 | CH₃ | trans phenyl-CH═CH— | —CH₂—CH₂— |
| 4 | i-propyl | benzothiophen-2-yl | —CH₂—CH₂— |
| 5 | i-propyl | trans phenyl-CH═CH— | —CH₂—CH₂— |
| 6 | CH₃ | trans phenyl-CH═CH— | —CH₂—CH₂—CH₂— |

and are named as:
N-hydroxy-4-(2-N-trans-cinnamoyl-N-hydroxyethylaminoethoxy)benzamide;
N-hydroxy-4-(2-N-trans-cinnamoyl-N-phenylaminoethoxy)benzamide;
N-hydroxy-4-(2-N-trans-cinnamoyl-N-methylaminoethoxy)benzamide;
N-hydroxy-4-(2-N-benzothiophen-2-yl-N-isopropylaminoethoxy)benzamide;
N-hydroxy-4-(2-N-trans-cinnamoyl-N-isopropylaminoethoxy)benzamide; and
N-hydroxy-4-(3-N-trans-cinnamoyl-N-methylaminopropoxy)benzamide.

Compounds of Formula (I) where R¹ and R³ are hydrogen, Ar¹ is phenyl, Ar² and Y are as defined in Table III below are:

TABLE III

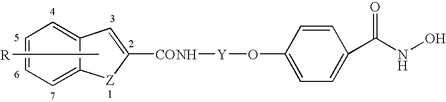

| Cpd | | | |
|---|---|---|---|
| # | Z | R | Y |
| 1 | S | H | —CH₂—CH₂— |
| 2 | O | H | —CH₂—CH₂— |
| 3 | NH | H | —CH₂—CH₂— |
| 4 | NMe | H | —CH₂—CH₂— |
| 5 | S | H | —CH₂—CH₂—CH₂— |
| 6 | O | H | —CH₂—CH₂—CH₂— |
| 7 | S | H | -(S)-CH(i-propyl)-CH₂— |
| 8 | S | H | -(S)-CH(ethyl)-CH₂— |
| 9 | S | H | -(S)-CH(methyl)-CH₂— |
| 10 | S | H | -(R)-CH(methyl)-CH₂— |
| 11 | O | H | -(S)-CH(ethyl)-CH₂— |
| 12 | S | H | -(R)-CH₂—CH(CH₃)— |
| 13 | S | H | -(S)-CH₂—CH(CH₃)— |
| 14 | O | H | -(R)-CH₂—CH(CH₃)— |
| 15 | S | 6-methoxy | —CH₂—CH₂— |
| 16 | S | 5-methyl | —CH₂—CH₂— |
| 17 | S | 3-chloro | —CH₂—CH₂— |
| 18 | O | 5-methyl | —CH₂—CH₂— |
| 19 | O | 6-methyl | —CH₂—CH₂— |
| 20 | S | 4-CF₃ | —CH₂—CH₂— |
| 21 | S | 5-fluoro | —CH₂—CH₂— |
| 22 | S | 5-methoxy | —CH₂—CH₂— |
| 23 | O | 5-chloro | —CH₂—CH₂— |
| 24 | O | 7-methoxy | —CH₂—CH₂— |
| 25 | O | 5-methoxy | —CH₂—CH₂— |
| 26 | O | 5-(2-methoxyethoxy)- | —CH₂—CH₂— |
| 27 | O | 5-(2-morpholin-4-ylethoxy)- | —CH₂—CH₂— |
| 28 | O | 5-pyridin-3-ylmethoxy | —CH₂—CH₂— |
| 29 | O | 3-methyl | —CH₂—CH₂— |
| 30 | S | 3 methyl | —CH₂—CH₂— |
| 31 | O | 5-(2-hydroxyethoxy)- | —CH₂—CH₂— |
| 32 | O | 5-(2-N,N-dimethylaminoethoxy)- | —CH₂—CH₂— |
| 33 | O | 6-CH₃OCH₂CH₂O | —CH₂—CH₂— |
| 34 | O | 6-(2-morpholin-4-ylethoxy)- | —CH₂—CH₂— |
| 35 | O | 6-pyridin-3ylmethoxy- | —CH₂—CH₂— |
| 36 | O | 3-ethyl | —CH₂—CH₂— |
| 37 | NH | 5-fluoro | —CH₂—CH₂— |
| 38 | NH | 5-methoxy | —CH₂—CH₂— |
| 39 | O | 3-CH₃OCH₂ | —CH₂—CH₂— |
| 40 | O | 3-phenoxymethyl | —CH₂—CH₂— |
| 41 | NH | 5,6-dimethoxy | —CH₂—CH₂— |
| 42 | O | 3-morpholino-4-ylmethyl | —CH₂—CH₂— |
| 43 | O | 3-N,N-dimethylaminomethyl | —CH₂—CH₂— |
| 44 | O | 3-i-propoxymethyl | —CH₂—CH₂— |
| 45 | O | 7-phenoxymethyl | —CH₂—CH₂— |
| 46 | O | 7-CH₂OCH₂ | —CH₂—CH₂— |
| 47 | O | 7-morpholino-4-ylmethyl | —CH₂—CH₂— |
| 48 | O | 7-N,N-dimethylaminomethyl | —CH₂—CH₂— |
| 49 | S | 5-methyl | —CH₂—CH₂—CH₂— |
| 50 | S | 6-methoxy | —CH₂—CH₂—CH₂— |
| 51 | O | 7-CH₃OCH₂ | —CH₂—CH₂—CH₂— |
| 52 | O | 7-phenoxymethyl | —CH₂—CH₂—CH₂— |
| 53 | O | 5-CH₃OCH₂CH₂O | -(R)-CH₂—CH(CH₃)— |
| 54 | O | H | (R)-CH(CH₃Smethyl)- |
| | | | CH₂— |
| 55 | O | H | (R)—CH(CH₃SO₂- |
| | | | methyl)-CH₂— |
| 56 | O | 3-(2-phenylethyl)- | —CH₂—CH₂— |
| 57 | O | 3-(N-methyl-N-benzylaminomethyl)- | —CH₂—CH₂— |
| 58 | O | 3-(N-methyl-N-2-phenylethyl- | —CH₂—CH₂— |
| | | aminomethyl)- | |
| 59 | O | 3-(3-hydroxypropylthiomethyl)- | —CH₂—CH₂— |
| 60 | O | 3-(3-hydroxypropylsulfinylmethyl)- | —CH₂—CH₂— |
| 61 | O | 3-(3-hydroxypropylsulfonylmethyl)- | —CH₂—CH₂— |
| 62 | O | 3-(N-methyl-N-2-indol-3-yl- | —CH₂—CH₂— |
| | | ethylaminomethyl)- | |
| 63 | O | 3-[2-(3-trifluoromethylphenyl)- | —CH₂—CH₂— |
| | | ethyl]- | |
| 64 | O | 3-[2-(3-trifluoromethoxyphenyl)— | —CH₂—CH₂— |
| | | ethyl]- | |
| 65 | O | 3-(N-hydroxyaminocarbonyl- | —CH₂—CH₂— |
| | | methylaminomethyl)- | |
| 66 | O | 3-(2-carboxyethylamino-methyl)- | —CH₂—CH₂— |
| 67 | O | H | (RS)-CH₂CH- |
| | | | (phenoxymethyl)- |
| 68 | O | 3-(3-hydroxypropyloxymethyl)- | —CH₂—CH₂— |
| 69 | O | 3-(2-fluorophenoxymethyl)- | —CH₂—CH₂— |
| 70 | O | 3-(3-fluorophenoxymethyl)- | —CH₂—CH₂— |
| 71 | O | 3-(4-fluorophenoxymethyl)- | —CH₂—CH₂— |
| 72 | O | 3-(2-methoxyethyloxymethyl)- | —CH₂—CH₂— |
| 73 | O | 3-(pyridin-4-yloxymethyl)- | —CH₂—CH₂— |
| 74 | O | 3-(2,4,6-trifluorophenoxymethyl)- | —CH₂—CH₂— |
| 75 | O | 3-(2-oxopyridin-1-ylmethyl)- | —CH₂—CH₂— |
| 76 | O | 3-(2,2,2-trifluoroethoxymethyl)- | —CH₂—CH₂— |
| 77 | O | 3-(4-imidazol-1-ylphenoxymethyl)- | —CH₂—CH₂— |
| 78 | O | 3-(4-[1.2.4]-triazin-1-ylphenoxy- | —CH₂—CH₂— |
| | | methyl)- | |
| 79 | O | 3-(pyrrolidin-1-ylmethyl)- | —CH₂—CH₂— |
| 80 | O | 3-(piperidin-1-ylmethyl)- | —CH₂—CH₂— |
| 81 | O | 3-(4-trifluoromethylpiperidin- | —CH₂—CH₂— |
| | | ylmethyl)- | |
| 82 | O | 3-(4-methylpiperazin-1-yl-methyl)- | —CH₂—CH₂— |
| 83 | O | 3-(3,3,3-trifluoropropyloxy-methyl)- | —CH₂—CH₂— |
| 84 | O | 4-methyl | —CH₂—CH₂— |
| 85 | O | 3-(4-fluorophenylthiomethyl)- | —CH₂—CH₂— |
| 86 | O | 3-(4-fluorophenylsulfinyl-methyl)- | —CH₂—CH₂— |
| 87 | O | 3-(4-fluorophenylsulfonyl-methyl)- | —CH₂—CH₂— |
| 88 | O | 3-(2,2,2-trifluoroethoxy-methyl)- | (S)-CH(ethyl)-CH₂— |
| 89 | O | 4-hydroxy | —CH₂—CH₂— |
| 90 | O | 5-chloro | (S)-CH(ethyl)-CH₂— |
| 91 | O | 5-chloro | (R)-CH₂—CH(methyl)- |
| 92 | O | 4-pyridin-3-ylmethyloxy-methyl | —CH₂—CH₂— |
| 93 | O | 4-methoxy | —CH₂—CH₂— |
| 94 | O | 4-(2-methoxyethyloxy)- | —CH₂—CH₂— |
| 95 | O | 4-pyridin-3-ylmethyloxy | —CH₂—CH₂— |
| 96 | NH | 4-methoxy | —CH₂—CH₂— |
| 97 | O | 3-(2-methoxyethyloxymethyl)- | (S)-CH(ethyl)-CH₂— |
| 98 | O | 3-(2-methoxyethyloxymethyl)- | (R)-CH₂—CH(methyl)- |
| 99 | O | 3-N,N-diethylaminomethyl | —CH₂—CH₂— |
| 100 | O | 5-(2-methoxyethyloxy)- | (S)-CH(ethyl)—CH₂— |
| 101 | O | 5-tetrahydropyran-4-yloxy | —CH₂—CH₂— |
| 102 | O | 5-tetrahydropyran-4-yloxy | (S)-CH(ethyl)-CH₂— |
| 103 | O | 5-tetrahydropyran-4-yloxy | (R)-CH₂—CH(methyl)- |
| 104 | O | 5-(2,2,2-trifluoroethyloxy)- | —CH₂—CH₂— |
| 105 | O | 5-(2-pyrrolidin-1-ylethyloxy)- | —CH₂—CH₂— |
| 106 | O | 5-(2-pyrrolidin-1-ylethyloxy)- | (S)-CH(ethyl)-CH₂— |
| 107 | O | 5-(2-pyrrolidin-1-ylethyloxy)- | (R)-CH₂—CH(methyl)- |
| 108 | O | 5-(piperidin-4-yloxy)- | —CH₂—CH₂— |
| 109 | O | H | (S)-CH(2-CH₃ethyl)- |
| | | | CH₂— |
| 110 | O | H | (S)—CH(2- |
| | | | CH₃SO₂ethyl)-CH₂— |

and are named as:
N-hydroxy-4-[2-(benzothiophen-2-ylcarbonylamino)ethoxy]-benzamide;
N-hydroxy-4-[2-(benzofuran-2-ylcarbonylamino)ethoxy]-benzamide;
N-hydroxy-4-[2-(1H-indol-2-ylcarbonylamino)ethoxy]-benzamide;
N-hydroxy-4-[2-(1-methylindol-2-ylcarbonylamino)ethoxy]-benzamide;
N-hydroxy-4-[3-(benzothiophen-2-ylcarbonylamino)propoxy]-benzamide;
N-hydroxy-4-[3-(benzofuran-2-ylcarbonylamino)propoxy]-benzamide;
N-hydroxy-4-[2S-(benzothiophen-2-ylcarbonylamino)-3-methylbutoxy]-benzamide;
N-hydroxy-4-[2S-(benzothiophen-2-ylcarbonylamino)butoxy]-benzamide;
N-hydroxy-4-[2S-(benzothiophen-2-ylcarbonylamino)-propoxy]-benzamide;
N-hydroxy-4-[2R-(benzothiophen-2-ylcarbonylamino)-propoxy]-benzamide;
N-hydroxy-4-[2S-(benzofuran-2-ylcarbonylamino)butoxy]-benzamide;
N-hydroxy-4-[2-(benzothiophen-2-ylcarbonylamino)-1R-methylethoxy]-benzamide;
N-hydroxy-4-[2-(benzothiophen-2-ylcarbonylamino)-1S-methylethoxy]-benzamide;
N-hydroxy-4-[2-(benzofuran-2-ylcarbonylamino)-1R-methylethoxy]-benzamide;
N-hydroxy-4-[2-(6-methoxybenzothiophen-2-ylcarbonylamino)ethoxy]-benzamide;
N-hydroxy-4-[2-(5-methylbenzothiophen-2-ylcarbonylamino)ethoxy]-benzamide;
N-hydroxy-4-[2-(3-chlorobenzothiophen-2-ylcarbonylamino)ethoxy]-benzamide;
N-hydroxy-4-[2-(5-methylbenzofuran-2-ylcarbonylamino)ethoxy]-benzamide;
N-hydroxy-4-[2-(6-methylbenzofuran-2-ylcarbonylamino)ethoxy]-benzamide;
N-hydroxy-4-[2-(4-trifluoromethylbenzothiophen-2-ylcarbonylamino)ethoxy]-benzamide;
N-hydroxy-4-[2-(5-fluorobenzothiophen-2-ylcarbonylamino)ethoxy]-benzamide;
N-hydroxy-4-[2-(5-methoxybenzothiophen-2-ylcarbonylamino)ethoxy]-benzamide;
N-hydroxy-4-[2-(5-chlorobenzofuran-2-ylcarbonylamino)ethoxy]-benzamide;
N-hydroxy-4-[2-(7-methoxybenzofuran-2-ylcarbonylamino)ethoxy]-benzamide;
N-hydroxy-4-[2-(5-methoxybenzofuran-2-ylcarbonylamino)ethoxy]-benzamide;
N-hydroxy-4-{2-[5-(2-methoxyethoxy)benzofuran-2-ylcarbonylamino]ethoxy}-benzamide;
N-hydroxy-4-{2-[5-(2-morpholin-4-ylethoxy)benzofuran-2-ylcarbonylamino]ethoxy}-benzamide;
N-hydroxy-4-{2-[5-(pyridin-3-ylmethoxy)benzofuran-2-ylcarbonylamino]ethoxy}-benzamide;
N-hydroxy-4-[2-(3-methylbenzofuran-2-ylcarbonylamino)ethoxy]-benzamide;
N-hydroxy-4-[2-(3-methylbenzothiophen-2-ylcarbonylamino)ethoxy]-benzamide;
N-hydroxy-4-{2-[5-(2-hydroxyethoxy)benzofuran-2-ylcarbonylamino]ethoxy}benzamide;
N-hydroxy-4-{2-[5-(2-N,N-dimethylaminoethoxy)benzofuran-2-ylcarbonylamino]-ethoxy}-benzamide;
N-hydroxy-4-{2-[6-(2-methoxyethoxy)benzofuran-2-ylcarbonylamino]ethoxy}-benzamide;
N-hydroxy-4-{2-[6-(2-morpholin-4-ylethoxy)benzofuran-2-ylcarbonylamino]ethoxy}-benzamide;
N-hydroxy-4-{2-[6-(pyridin-3-ylmethoxy)benzofuran-2-ylcarbonylamino]ethoxy}-benzamide;
N-hydroxy-4-[2-(3-ethylbenzofuran-2-ylcarbonylamino)ethoxy]-benzamide;
N-hydroxy-4-[2-(5-fluoroindol-2-ylcarbonylamino)ethoxy]-benzamide;
N-hydroxy-4-[2-(5-methoxyindol-2-ylcarbonylamino)ethoxy]-benzamide;
N-hydroxy-4-{2-[3-(methoxymethyl)benzofuran-2-ylcarbonylamino]ethoxy}benzamide;
N-hydroxy-4-{2-[3-(phenoxymethyl)benzofuran-2-ylcarbonylamino]ethoxy}benzamide;
N-hydroxy-4-[2-(5,6-dimethoxyindol-2-ylcarbonylamino)ethoxy]-benzamide;
N-hydroxy-4-f{2-[3-(morpholin-4-ylmethyl)benzofuran-2-ylcarbonylamino]ethoxy}-benzamide;
N-hydroxy-4-{2-[3-(N,N-dimethylaminomethyl)benzofuran-2-ylcarbonylamino]ethoxy}-benzamide;
N-hydroxy-4-{2-[3-(1-propoxymethyl)benzofuran-2-ylcarbonylamino]ethoxy}benzamide;
N-hydroxy-4-{2-[7-(phenoxymethyl)benzofuran-2-ylcarbonylamino]ethoxy}benzamide;
N-hydroxy-4-{2-[7-(methoxymethyl)benzofuran-2-ylcarbonylamino]ethoxy}benzamide;
N-hydroxy-4-{2-[7-(morpholin-4-ylmethyl)benzofuran-2-ylcarbonylamino]ethoxy}-benzamide;
N-hydroxy-4-{2-[7-(N,N-dimethylaminomethyl)benzofuran-2-ylcarbonylamino]ethoxy}-benzamide;
N-hydroxy-4-{3-[5-(methyl)benzothiophen-2-ylcarbonylamino]propoxy}-benzamide;
N-hydroxy-4-{3-[6-(methoxy)benzothiophen-2-ylcarbonylamino]propoxy}-benzamide;
N-hydroxy-4-{3-[7-(methoxymethyl)benzofuran-2-ylcarbonylamino]propoxy}-benzamide;
N-hydroxy-4-{3-[7-(phenoxymethyl)benzofuran-2-ylcarbonylamino]propoxy}-benzamide;
N-hydroxy-4-{2-[5-(2-methoxyethoxy)benzofuran-2-ylcarbonylamino]-1R-methyl ethoxy}benzamide.
N-hydroxy-4-(2R-benzofuran-2-ylcarbonylamino-3-methylthiopropoxy)benzamide;
N-hydroxy-4-(2R-benzofuran-2-ylcarbonylamino-3-methylsulfonylpropoxy)benzamide;
N-hydroxy-4-{2-[3-(2-phenylethyl)benzofuran-2-ylcarbonylamino]ethoxy}benzamide;
N-hydroxy-4-{2-[3-(N-methyl-N-benzylaminomethyl)benzofuran-2-ylcarbonylamino]-ethoxy}benzamide;
N-hydroxy-4-{2-[3-(N-methyl-N-2-phenylethylaminomethyl)benzofuran-2-ylcarbonylamino]-ethoxy}benzamide;
N-hydroxy-4-{2-[3-(3-hydroxypropylthiomethyl)benzofuran-2-ylcarbonylamino]-ethoxy}benzamide;
N-hydroxy-4-{2-[3-(3-hydroxypropylsulfinylmethyl)benzofuran-2-ylcarbonylamino]ethoxy}-benzamide;
N-hydroxy-4-{2-[3-(3-hydroxypropylsulfonylmethyl)benzofuran-2-ylcarbonylamino]ethoxy}-benzamide;
N-hydroxy-4-{2-[3-(N-methyl-N-2-indol-3-ylethylaminomethyl)benzofuran-2-ylcarbonylamino]-ethoxy}benzamide;
N-hydroxy-4-{2-[3-(2-(3-trifluoromethylphenyl)ethyl)benzofuran-2-ylcarbonylamino]-ethoxy}benzamide;
N-hydroxy-4-{2-[3-(2-(3-trifluoromethoxyphenyl)ethyl)benzofuran-2-ylcarbonylamino]-ethoxy}benzamide;
N-hydroxy-4-{2-[3-(N-hydroxyaminocarbonylmethylaminomethyl)benzofuran-2-ylcarbonyl-amino]ethoxy}benzamide;
N-hydroxy-4-{2-[3-(2-carboxyethylaminomethy)benzofuran-2-ylcarbonylamino]-ethoxy}-benzamide; and
N-hydroxy-4-[2-(benzofuran-2-ylcarbonylamino)-1RS-phenoxymethylethoxy}benzamide.
N-hydroxy-4-{2-[3-(3-hydroxypropoxymethyl)benzofuran-2-ylcarbonylamino]ethoxy}-benzamide;
N-hydroxy-4-{2-[3-(2-fluorophenoxymethyl)benzofuran-2-ylcarbonylamino]ethoxy}-benzamide;
N-hydroxy-4-{2-[3-(3-fluorophenoxymethyl)benzofuran-2-ylcarbonylamino]ethoxy}-benzamide;
N-hydroxy-4-{2-[3-(4-fluorophenoxymethyl)benzofuran-2-ylcarbonylamino]ethoxy}-benzamide;
N-hydroxy-4-{2-[3-(2-methoxyethyloxymethyl)benzofuran-2-ylcarbonylamino]ethoxy}-benzamide;
N-hydroxy-4-{2-[3-(pyridin-4-yloxymethyl)benzofuran-2-ylcarbonylamino]ethoxy}-benzamide;
N-hydroxy-4-{2-[3-(2,4,6-trifluorophenoxymethyl)benzofuran-2-ylcarbonylamino]-ethoxy}benzamide;
N-hydroxy-4-{2-[3-(2-oxopyridin-1-ylmethyl)benzofuran-2-ylcarbonylamino]ethoxy}-benzamide;
N-hydroxy-4-{2-[3-(2,2,2-trifluoroethoxymethyl)benzofuran-2-ylcarbonylamino]-ethoxy}benzamide;
N-hydroxy-4-{2-[3-(4-imidazol-1-ylphenoxymethyl)benzofuran-2-ylcarbonylamino]-ethoxy}benzamide;
N-hydroxy-4-{2-[3-(4-[1.2.4]-triazin-1-ylphenoxymethyl)benzofuran-2-ylcarbonyl-amino]ethoxy}benzamide;
N-hydroxy-4-{2-[3-(pyrrolidin-1-methyl)benzofuran-2-ylcarbonylamino]ethoxy}-benzamide;
N-hydroxy-4-{2-[3-(piperidin-1-methyl)benzofuran-2-ylcarbonylamino]ethoxy}-benzamide;
N-hydroxy-4-{2-[3-(4-trifluoromethylpiperidin-1-methyl)benzofuran-2-ylcarbonylamino]-ethoxy}benzamide;
N-hydroxy-4-{2-[3-(4-methylpiperazin-1-methyl)benzofuran-2-ylcarbonylamino]-ethoxy}benzamide;
N-hydroxy-4-{2-[3-(3,3,3-trifluoropropyloxymethyl)benzofuran-2-ylcarbonylamino]-ethoxy}benzamide;
N-hydroxy-4-[2-(4-methylbenzofuran-2-ylcarbonylamino)-ethoxy]benzamide;
N-hydroxy-4-{2-[3-(4-fluorophenylthiomethyl)benzofuran-2-ylcarbonylamino]-ethoxy}-benzamide;
N-hydroxy-4-{2-[3-(4-fluorophenylsulfinylmethyl)benzofuran-2-ylcarbonylamino]-ethoxy}-benzamide;
N-hydroxy-4-{2-[3-(4-fluorophenylsulfonylmethyl)benzofuran-2-ylcarbonylamino]-ethoxy}-benzamide;
N-hydroxy-4-{2S-[3-(2,2,2-trifluoroethoxymethyl)benzofuran-2-ylcarbonylamino]-butoxy}-benzamide;
N-hydroxy-4-[2-(4-hydroxybenzofuran-2-ylcarbonylamino)ethoxy]benzamide;
N-hydroxy-4-[2S-(5-chlorobenzofuran-2-ylcarbonylamino)butoxy]benzamide;
N-hydroxy-4-[2-(5-chlorobenzofuran-2-ylcarbonylamino]-1R-methylethoxy]benzamide;
N-hydroxy-4-[2-(4-pyridin-3-ylmethyloxymethylbenzofuran-2-ylcarbonylamino)-ethoxy]benzamide;
N-hydroxy-4-[2-(4-methoxybenzofuran-2-ylcarbonylamino)ethoxy]benzamide;
N-hydroxy-4-{2-[4-(2-methoxyethyloxy)benzofuran-2-ylcarbonylamino)ethoxy}-benzamide;
N-hydroxy-4-[2-(4-pyridin-3-ylmethyloxybenzofuran-2-ylcarbonylamino)-ethoxy]benzamide;
N-hydroxy-4-[2-(4-methoxyindol-2-ylcarbonylamino)ethoxy]benzamide;
N-hydroxy-4-{2S-[3-(2-methoxyethyloxymethyl)benzofuran-2-ylcarbonylamino]-butoxy}benzamide;
N-hydroxy-4-{2-[3-(2-methoxyethyloxymethyl)benzofuran-2-ylcarbonylamino]-1R-methyl-ethoxy}benzamide;
N-hydroxy-4-{2-[3-(N,N-diethylaminomethyl)benzofuran-2-ylcarbonylamino]ethoxy}-benzamide;
N-hydroxy-4-{2S-[5-(2-methoxyethyloxy)benzofuran-2-ylcarbonylamino]butoxy}-benzamide;
N-hydroxy-4-{2-[5-(tetrahydropyran-4-yloxy)benzofuran-2-ylcarbonylamino]ethoxy}-benzamide;
N-hydroxy-4-{2S-[5-(tetrahydropyran-4-yloxy)benzofuran-2-ylcarbonylamino]butoxy}-benzamide;
N-hydroxy-4-{2-[5-(tetrahydropyran-4-yloxy)benzofuran-2-ylcarbonylamino]-1R-methyl-ethoxy}benzamide;
N-hydroxy-4-{2-[5-(2,2,2-trifluoroethyloxy)benzofuran-2-ylcarbonylamino]ethoxy}-benzamide;
N-hydroxy-4-{2-[5-(2-pyrrolidin-1-ylethyloxy)benzofuran-2-ylcarbonylamino]ethoxy}-benzamide;
N-hydroxy-4-{2S-[5-(2-pyrrolidin-1-ylethyloxy)benzofuran-2-ylcarbonylamino]butoxy}-benzamide;
N-hydroxy-4-{2-[5-(2-pyrrolidin-1-ylethyloxy)benzofuran-2-ylcarbonylamino]-1R-methyl-ethoxy}benzamide;
N-hydroxy-4-{2-[5-(piperidin-4-yloxy)benzofuran-2-ylcarbonylamino]ethoxy}-benzamide;
N-hydroxy-4-[2S-(benzofuran-2-ylcarbonylamino)-4-methylthiobutoxy]benzamide; and
N-hydroxy-4-[2S-(benzofuran-2-ylcarbonylamino)-4-methylsulfonylbutoxy]benzamide.

Compounds of Formula (I) where R¹ and R³ are hydrogen, Ar¹ is isoxazol-5-yl and Ar² and Y are as defined in Table IV below are:

TABLE IV

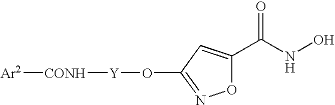

| Cpd | | |
|---|---|---|
| # | Ar² | Y |
| 1 | 4-biphenyl | (R)-CH₂—CH(CH₃)— |
| 2 | 4-biphenyl | (S)-CH(ethyl)—CH₂— |
| 3 | benzofuran-2-yl | —CH₂—CH₂— |
| 4 | trans phenyl-CH═CH— | —CH₂—CH₂— |
| 5 | 4-(2-ethoxyphenyl)phenyl | —CH₂—CH₂—CH₂— |
| 6 | 3-biphenyl | —CH₂—CH₂—CH₂— |
| 7 | 4-biphenyl | —CH₂—CH₂—CH₂— |
| 8 | naphth-2-yl | —CH₂—CH₂— |
| 9 | 3-methylbiphen-4-yl | —CH₂—CH₂— |
| 10 | 2′-ethoxybiphen-4-yl | —CH₂—CH₂— |
| 11 | 3-methylbiphen-4-yl | —CH₂—CH₂—CH₂— |
| 12 | 4-phenylthiazol-2-yl | —CH₂—CH₂—CH₂— |
| 13 | naphth-2-yl | —CH₂—CH₂—CH₂— |
| 14 | naphth-1-yl | —CH₂—CH₂—CH₂— |
| 15 | 2-(2-phenylethyl)phenyl | —CH₂—CH₂—CH₂— |
| 16 | naphth-1-yl | —CH₂—CH₂— |
| 17 | benzofuran-2-yl | (S)-CH(ethyl)-CH₂— |
| 18 | 3-biphenyl | (S)-CH(ethyl)-CH₂— |
| 19 | benzofuran-2-yl | (R)-CH₂—CH(methyl)- |
| 20 | 3-biphenyl | (R)-CH₂—CH(methyl)- |
| 21 | 3-biphenyl | —CH₂—CH₂— |
| 22 | 4-biphenyl | —CH₂—CH₂— |
| 23 | 4-phenylthiazol-2-yl | —CH₂—CH₂— |
| 24 | 2-(2-phenylethyl)phenyl | —CH₂—CH₂— |
| 25 | 2-biphenyl | —CH₂—CH₂— |
| 26 | 2-biphenyl | —CH₂—CH₂—CH₂— |
| 27 | naphth-2-yl | (S)-CH(ethyl)-CH₂— |
| 28 | naphth-1-yl | (S)-CH(ethyl)-CH₂— |
| 29 | naphth-2-yl | (R)-CH₂—CH(methyl)- |
| 30 | naphth-1-yl | (R)-CH₂—CH(methyl)- |
| 31 | benzofuran-2-yl | —CH₂—CH₂—CH₂— |
| 32 | trans phenylCH═CH— | —CH₂—CH₂—CH₂— |
| 33 | 3-(phenoxymethyl)benzofuran-2-yl | —CH₂—CH₂— |

and are named as:
N-hydroxy-3-[2-(biphen-4-ylcarbonylamino)-1R-methylethoxy]isoxazol-5-ylcarboxamide;
N-hydroxy-3-[2S-(biphen-4-ylcarbonylamino)butoxy]isoxazol-5-ylcarboxamide;
N-hydroxy-3-[2-(benzofuran-2-ylcarbonylamino)ethoxy]isoxazol-5-ylcarboxamide;
N-hydroxy-3-[2-(trans-cinnanoylamino)ethoxy]isoxazol-5-ylcarboxamide;
N-hydroxy-3-[3-(4-(2-ethoxyphenyl)phenylcarbonylamino)propoxy]isoxazol-5-ylcarboxamide;
N-hydroxy-3-[3-(biphen-3-ylcarbonylamino)propoxy]isoxazol-5-ylcarboxamide;
N-hydroxy-3-[3-(biphen-4-ylcarbonylamino)propoxy]isoxazol-5-ylcarboxamide;
N-hydroxy-3-[2-(naphth-2-ylcarbonylamino)ethoxy]isoxazol-5-ylcarboxamide;
N-hydroxy-3-[2-(3-methylbiphen-4-ylcarbonylamino)ethoxy]isoxazol-5-ylcarboxamide;
N-hydroxy-3-[2-(2′-ethoxylbiphen-4-ylcarbonylamino)ethoxy]isoxazol-5-ylcarboxamide;
N-hydroxy-3-[3-(3-methylbiphen-4-ylcarbonylamino)propoxy]isoxazol-5-ylcarboxamide;
N-hydroxy-3-[3-(4-phenylthiazol-2-ylcarbonylamino)propoxy]isoxazol-5-ylcarboxamide;
N-hydroxy-3-[3-(naphth-2-ylcarbonylamino)propoxy]isoxazol-5-ylcarboxamide;
N-hydroxy-3-[3-(naphth-1-ylcarbonylamino)propoxy]isoxazol-5-ylcarboxamide;
N-hydroxy-3-{3-[2-(2-phenylethyl)phenylcarbonylamino]propoxy}isoxazol-5-ylcarboxamide;
N-hydroxy-3-[2-(naphth-1-ylcarbonylamino)ethoxy]isoxazol-5-ylcarboxamide;
N-hydroxy-3-[2S-(benzofuran-2-ylcarbonylamino)butoxy]isoxazol-5-ylcarboxamide;
N-hydroxy-3-[2S-(biphen-3-ylcarbonylamino)butoxy]isoxazol-5-ylcarboxamide;
N-hydroxy-3-[2-(benzofuran-2-ylcarbonylamino)-1R-methylethoxy]isoxazol-5-ylcarboxamide;
N-hydroxy-3-[2-(biphen-3-ylcarbonylamino)-1R-methylethoxy]isoxazol-5-ylcarboxamide;
N-hydroxy-3-[2-(biphen-3-ylcarbonylamino)ethoxy]isoxazol-5-ylcarboxamide;
N-hydroxy-3-[2-(biphen-4-ylcarbonylamino)ethoxy]isoxazol-5-ylcarboxamide;
N-hydroxy-3-[2-(4-phenylthiazol-2-ylcarbonylamino)ethoxy]isoxazol-5-ylcarboxamide;
N-hydroxy-3-{2-[2-(2-phenylethyl)phenylcarbonylamino]ethoxy}isoxazol-5-ylcarboxamide;
N-hydroxy-3-[2-(biphen-2-ylcarbonylamino)ethoxy]isoxazol-5-ylcarboxamide;
N-hydroxy-3-[3-(biphen-2-ylcarbonylamino)propoxy]isoxazol-5-ylcarboxamide;
N-hydroxy-3-[2S-(naphth-2-ylcarbonylamino)butoxy]isoxazol-5-ylcarboxamide;
N-hydroxy-3-[2S-(naphth-1-ylcarbonylamino)butoxy]isoxazol-5-ylcarboxamide;
N-hydroxy-3-[2-(naphth-2-ylcarbonylamino)-1R-methylethoxy]isoxazol-5-ylcarboxamide;
N-hydroxy-3-[2-(naphth-1-ylcarbonylamino)-1R-methylethoxy]isoxazol-5-ylcarboxamide;
N-hydroxy-3-[3-(benzofuran-2-ylcarbonylamino)propoxy] isoxazol-5-ylcarboxamide;
N-hydroxy-3-[3-(trans-cinnamoylamino)propoxy]isoxazol-5-ylcarboxamide; and
N-hydroxy-3-[2-(3-phenoxymethylbenzofuran-2-ylcarbonylamino)ethoxy]isoxazol-5-yl-carboxamide.

Representative compounds of Formula (II) which can be used to practice the Invention are disclosed in WO 05/019174 in Tables I-III and are incorporated herein by reference.

### General Synthesis

Compounds of this invention can be made by the methods depicted in the reaction scheme shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula (I) where X is —O— or —S(O)ₙ— where n is 0 to 2 and other groups are as described in the Summary of the Invention can be prepared by the procedure illustrated and described in Scheme A below.

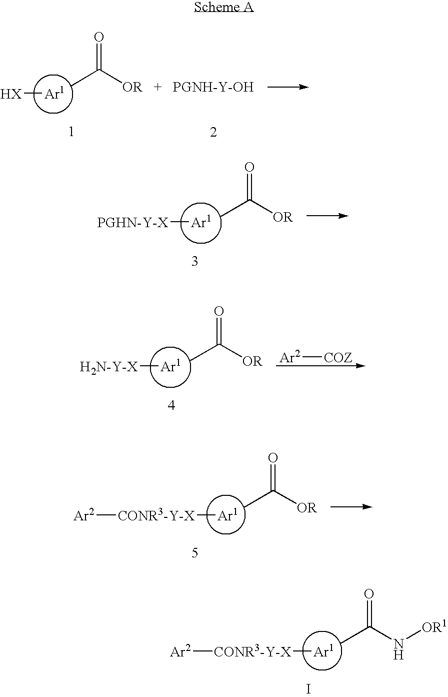

Reaction of a compound of formula 1 where R is alkyl, X is —O— or —S— and Ar¹ is as defined in the Summary of the Invention with an aminoalcohol of formula 2 where PG is a suitable amino protecting group provides a compound of formula 3. The reaction is carried out in the presence of triphenylphosphine and diisopropyl azodicarboxylate in a suitable organic solvent such as tetrahydrofuran, and the like.

Compounds of formula 1 such as methyl 4-hydroxybenzoate, methyl 4-mercaptobenzoate, and methyl 3-hydroxyisoxazole-5-carboxylate are commercially available. Compounds of formula 2 can be prepared from commercially available aminoalcohols by reacting the amine with a suitable amino protecting group such as benzyloxycarbonyl, tert-butoxycarbonyl and the like under reaction conditions well known in the art. A detailed description of suitable amino protecting groups and reaction conditions for their preparation can be found in T. W. Greene, Protecting Groups in Organic Synthesis, John Wiley & Sons, Inc. 1981 the teaching of which is incorporated herein by reference in its entirety. Aminoalcohols such as 2-ethanolamine, 2-amino-1-propanol, 2-methylaminoethanol, 2-amino-2-methyl-1-propanol, 2-amino-1-propanol, 4-amino-2-butanol, and 1-amino-2-butanol are commercially available. Alternatively, compounds of formula 2 can be prepared from commercially available aminoacids by protecting the amino group with a suitable protecting group followed by reduction of the acid group to the hydroxy group with a suitable reducing agent under conditions well known in the art. If compounds of Formula (I) where X is —SO₂— are desired, the corresponding compound of formula 3 where X is —S— can be treated with an oxidizing agent such as OXONE®, m-chloroperbenzoic acid, and the like.

Removal of the amino protecting group in 3 provides a compound of formula 4. The reaction conditions employed for removal of the amino protecting group depend on the nature of the protecting group. For example, if the protecting group is tert-butoxycarbonyl, it is removed under acid reaction conditions. Suitable acids are trifluoroacetic acid, hydrochloric acid, and the like in a suitable organic solvent such as methanol, dioxane, tetrahydrofuran, and the like. If the protecting group is benzyl or benzyloxycarbonyl, it is removed under catalytic hydrogenation reaction conditions. Suitable catalyst are palladium based catalysts and others known in the art. Other suitable reaction conditions for their removal can be found in T. W. Greene, Protecting Groups in Organic Synthesis, John Wiley & Sons, Inc. 1981. The reaction is carried out in an inert organic solvent methylene chloride, tetrahydrofuran, dioxane, and the like.

Reaction of 4 with an acid or acid derivative (e.g., acid halide) of formula Ar²—COZ where Z is hydroxy or halo provides a compound of formula 5. Again, the reaction conditions employed depend on the nature of the Z group. If Z is hydroxy, the reaction is typically carried out in the presence of a suitable coupling agent e.g., benzotriazole-1-yloxytrispyrrolidino-phosphonium hexafluorophosphate (PyBOP®), O-benzotriazol-1-yl-N,N,N′,N′-tetramethyl-uronium hexafluorophosphate (HBTU), O-(7-azabenzotriazol-1-yl)-1,1,3,3-tetramethyl-uronium hexafluorophosphate (HATU), 1-(3-dimethylaminopropyl)-3-ethylcarbodiimide hydrochloride (EDC. HCl), or 1,3-dicyclohexylcarbodiimide (DCC), optionally in the presence of 1-hydroxybenzotriazole hydrate (HOBT. H₂O), and a base such as N,N-diisopropylethylamine, triethylamine, N-methylmorpholine, and the like. The reaction is typically carried out at 20 to 30° C., preferably at about 25° C., and requires 2 to 24 hours to complete. Suitable reaction solvents are inert organic solvents such as halogenated organic solvents (e.g., methylene chloride, chloroform, and the like), acetonitrile, N,N-dimethylformamide, ethereal solvents such as tetrahydrofuran, dioxane, and the like. Preferably, the reaction is carried out with HOBt. H₂O, EDC.HCl in dichloromethane or N,N-dimethylformamide.

When Ar²COZ is an acid halide, the reaction is carried out in the presence of a suitable base (e.g. triethylamine, diisopropylethylamine, pyridine, and the like). Suitable reaction solvents are polar organic solvents such as tetrahydrofuran, acetonitrile, N,N-dimethylformamide (DMF), dichloromethane, or any suitable mixtures thereof. The acid halide such as acid chloride can be prepared by reacting the corresponding acids with an halogenating agent such as oxalyl chloride, thionyl chloride, phosphorus oxychloride, and the like. Acids of formula Ar²COZ are either commercially available or they can be prepared from commercially available starting materials by methods known in the art. For example, benzoic acid, cinnamic acid, phenylacetic acid, nicotinic acid, isonicotinic acid, 3-methylbenzofuran-2-carboxylic acid, and benzofuran-2-carboxylic acid are commercially available. Others such as 3-phenoxymethylbenzofuran-2-carboxylic acid can be readily prepared from commercially available 3-methylbenzofuran-2-carboxylic acid by first converting it to 2-bromomethylbenzofuran-2-carboxylic acid (brominating it with N-bromosuccinimide under conditions well known in the art) followed by reacting with phenol. Compound 5 where R³ is hydrogen can optionally be converted to a corresponding compound of formula 5 where R³ is other than hydrogen by reacting it with an alkylating agent under conditions well known in the art.

Compound 5 is then converted to a compound of Formula (I) by reacting it with aqueous hydroxylamine in the presence of a base such as sodium hydroxide and a mixture of organic solvents such as tetrahydrofuran and methanol. Alternatively, the acid group in 5 is first activated with a suitable coupling agent such as 1-(3-dimethylaminopropyl)-3-ethylcarbodiimide hydrochloride (EDC. HCl), or 1,3-dicyclohexylcarbodiimide (DCC), optionally in the presence of 1-hydroxybenzotriazole hydrate (HOBT. H₂O) in a suitable organic solvent such as dimethylformamide, and the like, and then reacted with hydroxylamine hydrochloride in the presence of a base such as N,N-diisopropylethylamine, triethylamine, N-methylmorpholine, and the like. Compounds of Formula (I) can also be prepared from compound 5 by the methods disclosed in U.S. Pat. No. 5,998,412, the disclosure of which is incorporated herein by reference in its entirety.

A compound of Formula (I) can be converted to other compounds of Formula (I). For example, a compound of Formula (I) where Ar¹ is phenylene, X is —O—, Y is ethylene, Ar² is 3-dimethylaminomethylbenzofuran-2-yl, R¹ and R³ are hydrogen can be prepared by reacting a compound of formula 4 where Ar¹ is phenylene, X is —O—, Y is ethylene, and R is alkyl with 3-methylbenzofuran-2-carboxylic acid as described above to give a compound of formula 5 where Ar² is 3-methylbenzofuran-2-yl. Bromination of the methyl group with a suitable brominating agent such as N-bromosuccinimide, followed by reaction with dimethylamine provides the corresponding 3-dimethylaminobenzofuran-2-yl compound which is then converted to the desired compound under the reaction conditions described above.

A compound of Formula (II) where Z¹ is —CONR^{3a}—, —SO₂NR⁵—, —NR⁷CONR⁸—, —NR⁹SO₂NR¹⁰—, or —OCONR¹¹— and other groups are as defined in the Summary of the Invention can be prepared by the procedure illustrated and described in Scheme B below.

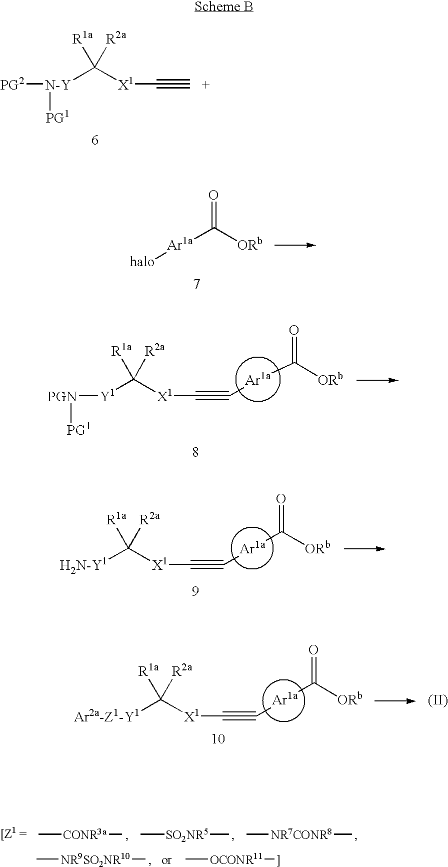

Reaction of an alkyne of formula 6 (where PG² is a suitable amino protecting group and PG¹ is hydrogen or a suitable amino protecting group such as tert-butoxycarbonyl, benzyloxycarbonyl, and the like, more preferably tert-butoxycarbonyl), with a compound of formula 7 (where R^{b} is alkyl, preferably methyl or ethyl and the halo group is preferably iodo), provides a compound of formula 8. The reaction is carried out in a suitable organic solvent such as dimethylformamide and in the presence of an organic base such as triethylamine, and the like and a suitable catalyst such as PdCl₂(Ph₃P)₂, and the like.

Compounds of formula 6 can be prepared from commercially available starting materials by methods well known in the art. For example, N-tert-butoxycarbonyl-propargylamine can be prepared by reacting commercially available propargylamine with tert-butoxycarbonyl anhydride in the presence of a base such as triethylamine, ethylisopropylamine, and the like. Compounds of formula 6 can also be prepared from N-protected amino acids by first reducing the acid group to give the corresponding aldehyde by methods well known in the art and then reacting the aldehyde with Ohira reagent (see Ohira, S. Synth. Commun., 19, 561-564, (1989)) to give a compound of formula 6. Amino acids suitable for preparing compounds of formula 6 are commercially available. For example, 1-amino-cyclobutanecarboxylic acid, homoalanine, aspartic acid, gamma-n-butyric acid, 3-amino-3-phenylpropionic acid, 4-amino-2,2-dimethyl butyric acid, piperidine-3-carboxylic acid, 4-aminopiperidine-4-carboxylic acid, 4-amino-4-carboxytetrahydropyran, 2-aminocyclopentaneacetic acid, and 2-amino-1-cyclopentanecarboxylic acid are commercially available. Alpha and beta amino acids can also be prepared by methods described in Duthaler, R. O. Tetrahedron, 50, 1539-1650 (1994) and Cole, D. C. Tetrahedron, 50, 9517-9582, (1994), the disclosures of which are incorporated herein by reference in their entirety.

Compounds of formula 6 where PG¹ is an amino protecting group can also be prepared from commercially available alcohols under Mitsunobu reaction conditions.

Removal of the amino-protecting group provides a compound of formula 9. The reaction conditions employed depend on the nature of the protecting group. For example, if the amino-protecting group is tert-butoxycarbonyl, it is removed by treating a compound of formula 8 with an acid such as trifluoroacetic acid, hydrochloric acid, and the like, in a suitable organic solvent such as dioxane, tetrahydrofuran, methanol, dichloromethane, and the like.

Compounds of formula 7 are either commercially available or they can be prepared by methods well known in the art. For example, methyl 4-iodobenzoate is commercially available. 4-Iodo-2-thiophenecarboxylic methyl ester can be prepared from commercially available 4-iodo-2-thiophenecarboxylic acid under standard esterification reaction conditions.

Compound 9 is then converted to a compound of formula 10 where Z¹ is —CONR^{3a}—, —SO₂NR⁵—, —NR⁷CONR⁸—, —NR⁹SO₂NR¹⁰—, or —OCONR¹¹— by methods well known in the art. Some such methods are described below.

A compound of formula 10 where Z¹ is —CONH— or —SO₂NH— is prepared by reacting a compound of formula 9 with an acylating or sulfoylating agent of formula Ar^{2a}COL or Ar^{2a}SO₂L respectively, where L is a leaving group under acylating or sulfonylating reaction conditions such as halo (particularly chloro or bromo). Suitable for solvents for the reaction include organic solvents such as dichloromethane, tetrahydrofuran, dioxane, dimethylformamide, and the like. The reaction is carried out in the presence of an organic base such as triethyamine, pyridine, and the like. Acylating or sulfonylating agent of formula Ar^{2a}COL or Ar^{2a}SO₂L are either commercially available or they can be readily prepared by methods well known in the art. For example, Ar^{2a}COL can be prepared by reacting the corresponding acids with a halogenating agent such as oxalyl chloride, thionyl chloride, and the like.

Alternatively, a compound of Formula (II) when Z¹ is —CONH— can be prepared by heating 9 with an acid anhydride. Suitable solvents for the reaction are tetrahydrofuran, dioxane, and the like.

Alternatively, a compound of Formula (II) when Z¹ is —CONH— can be prepared by reacting an acid of formula Ar^{2a}—COOH in the presence of a suitable coupling agent e.g., benzotriazole-1-yloxytrispyrrolidinophosphonium hexafluorophosphate (PyBOP®), O-benzotriazol-1-yl-N,N,N′,N′-tetramethyl-uronium hexafluorophosphate (HBTU), O-(7-azabenzotriazol-1-yl)-1,1,3,3-tetramethyl-uronium hexafluorophosphate (HATU), 1-(3-dimethylaminopropyl)-3-ethylcarbodiimide hydrochloride (EDC.HCl), or 1,3-dicyclohexylcarbodiimide (DCC), optionally in the presence of 1-hydroxybenzotriazole hydrate (HOBt.H₂O), and a base such as N,N-diisopropylethylamine, triethylamine, N-methylmorpholine, and the like. The reaction is typically carried out at 20 to 30° C., preferably at about 25° C., and requires 2 to 24 h to complete. Suitable reaction solvents are inert organic solvents such as halogenated organic solvents (e.g., methylene chloride, chloroform, and the like), acetonitrile, dimethylformamide, ethereal solvents such as tetrahydrofuran, dioxane, and the like. Acids of formula Ar^{2a}—COOH such as benzoic acid, cinnamic acid, phenylacetic acid, nicotinic acid, isonicotinic acid, and benzofuran-2-carboxylic acid are commercially available. Others such as 3-phenoxymethylbenzofuran-2-carboxylic acid can be readily prepared from commercially available 3-methylbenzofuran-2-carboxylic acid by first converting it to 2-bromomethylbenzofuran-2-carboxylic acid (brominating it with N-bromosuccinimide under conditions well known in the art) followed by reacting with phenol.

A compound of formula 10 where Z¹ is —NR⁷CONH— is prepared by reacting a compound of formula 9 with an activating agent such as carbonyl diimidazole, followed by displacement of the imidazole group with a primary or secondary amine of formula Ar^{2a}NHR⁷. Suitable reaction solvents include tetrahydrofuran, dioxane, and the like.

Alternatively, a compound of formula 10 where Z¹ is —NR⁷CONH— is prepared by reacting compound 9 with a carbamoyl halide of formula Ar^{2a}NR⁷COL or an isocyanate of formula Ar^{2a}N═C═O under conditions well known in the art.

A compound of formula 10 where Z¹ is —NR⁹SO₂NH— is prepared by reacting a compound of formula 9 with a sulfamoyl halide of formula Ar^{2a}NR⁹SO₂L under reaction conditions described above. Sulfamoyl halides are either commercially available or may be prepared by methods such as those described in Graf, R., German Patent 931225 and Catt, J. D. and Matler, W. L., J. Org. Chem., 1974, 39, 577-568.

A compound of formula 10 where Z¹ is —OC(O)NH— is prepared by reacting a compound of formula 9 with acylating agent of formula Ar^{2a}OC(O)L under reaction conditions described above.

Compound 10 is then converted to a compound of Formula (II) by reacting it with aqueous hydroxylamine in the presence of a base such as sodium hydroxide and a mixture of organic solvents such as tetrahydrofuran and methanol.

A compound of Formula (II) can be converted to other compounds of Formula (II). For example, a compound of Formula (II) where any of R^{3a}-R¹¹ is alkyl can also be prepared by reacting a corresponding compound of Formula (II) where any of R^{3a}-R¹¹ is hydrogen with an alkylating agent under conditions well known in the art. Other methods of preparing compounds of Formula (II) from compound 10 are analogous to the methods disclosed in U.S. Pat. No. 5,998,412 the disclosure of which is incorporated herein by reference in its entirety.

Compounds of Formula (II) where Z¹ is —NR¹²COO— can be prepared by following the procedures described above, by using starting materials such as 3-butyn-2-ol, 3-butyn-1-ol and 4-pentyn-2-ol.

### General Procedures for the Preparation of Anti-γ-H2AX Antibody and Detection of γ-H2AX:

The isolated or purified antibody or antigenically-reactive fragment thereof can be derived from any animal. The particular animal from which the antibody or fragment thereof is derived is not essential to the present invention. The antibody or fragment thereof may be humanized, meaning that an antibody, originally derived from an animal, is altered by substituting amino acids not involved in antigen binding with amino acids from corresponding regions of a human immunoglobulin. The use of humanized antibodies or fragments thereof limits the antigenicity of a foreign antibody.

Isolated or purified antibodies or antigenically-reactive fragments thereof directed to γ-H2AX can be generated using various methods well-known in the art. For example, anti-γ-H2AX antibodies can be isolated or purified from serum taken from an animal immunized with γ-H2AX. Immunization may be accomplished using standard procedures. The immunizing peptide can be an intact γ-H2AX peptide or a peptide fragment which comprises the C-terminal phosphorylated serine and which is recognizable by an antibody. Similarly, the peptide can be isolated or purified from an organism or synthetically made using methods known in the art. The immunizing peptide can be administered alone, or in a composition further comprising an adjuvant, such as complete or incomplete Freund's adjuvant.

In order to ensure that the appropriate antibody or fragment thereof is produced in the immunized animal, blood is taken between immunizations and the serum is assayed for γ-H2AX binding specificity. Binding specificity can be determined using an immunoassay such as, for example, ELISA. The antibody or antigenically-reactive fragment thereof may be isolated from the serum by any of a number of separation techniques used in the art, such as, for example, affinity, ion exchange, gel filtration, hydrophobic interaction, and/or protein A affinity chromatography (Harlow et al., Antibodies, A Laboratory Manual, Cold Spring Harbor Laboratory Press, Cold Spring, N.Y. (1988)).

Alternatively, monoclonal antibodies may be produced using hybridoma cells. Monoclonal antibodies are an homogenous population of a single antibody clone with defined specificity toward one epitope on an antigen, i.e. the C-terminal phosphorylated serine of an γ-H2AX histone protein. The technology for producing monoclonal antibodies is well known (Harlow et al. (1988), supra; and, in general, Roitt et al., Immunology, 4.sup.th Ed., Mosby, London, England (1996)). Briefly, an animal is immunized with an antigen, i.e., γ-H2AX. Lymphocytes are isolated from the spleen or lymph nodes of the immunized animal. Preferably, lymphocytes for preparation of monoclonal antibodies are taken from animals which have demonstrated production of the appropriate antibody. These lymphocytes are fused with an immortal cell line and successfully fused cells are selected for by culturing in HAT medium. The culture supernatants of the resulting hybridoma cells can subsequently be screened for γ-H2AX specific antibodies using methods well known in the art e.g., in U.S. Pat. No. 6,362,317. Culture supernatant containing anti-γ-H2AX is collected and the antibody is isolated and purified.

Isolated or purified antibodies or antigenically-reactive fragments thereof of the present invention also can be produced by recombinant techniques (Sambrook et al., Molecular Cloning, 2.sup.nd Ed., Cold Spring Harbor Laboratory Press, Cold Spring, N.Y. (1989)). For example, recombinant antibodies can be produced by cloning cDNA encoding anti-γ-H2AX. The cDNA encoding the appropriate light and heavy chains is incorporated into an expression vector and introduced into a host cell. One of ordinary skill in the art will appreciate that the particular expression vector and regulatory sequences used are not limited as long as the appropriate peptides are produced in the host cell, whether eukaryotic or prokaryotic, and are able to specifically bind γ-H2AX. One of ordinary skill in the art will further appreciate that antigenically-reactive fragments also can be produced in this manner. In either case, the expression products are screened for binding specificity using routine methods. Antigenically-reactive antibody fragments also can be generated chemically, e.g., by cleaving an antibody with a protease, such as, for example, pepsin or papain.

In order to detect binding to an γ-H2AX protein, the isolated or purified antibody or antigenically-reactive fragment thereof is, preferably, labeled with a means of facilitating detection. By “means of facilitating detection” is meant that an antibody or fragment thereof is associated with a substrate detectable by conventional, i.e., spectroscopic, biochemical, immunochemical, photochemical or chemical, means. As such, the isolated or purified antibody or antigenically-reactive fragment thereof is preferably labeled with, for example, an enzyme, a radioactive isotope, biotin or a fluorescent molecule, such as fluorescein or rhodamine. Labels may be complexed with the antibody or antigenically-reactive fragment thereof by any means known in the art. For example, a means of detection, such as an enzyme, is biotinylated while the antibody is associated with streptavidin. Biotin and streptavidin bind each other, thereby attaching the label to the antibody. Alternatively, a means of detection can be linked to the antibody via covalent bonding.

Similarly, the present invention provides for fusion proteins comprising an isolated or purified antibody or antigenically-reactive fragment thereof which specifically binds to the C-terminal phosphorylated serine in an γ-H2AX histone protein. The isolated or purified antibody or fragment thereof can be fused to an effector protein such as, for example, a toxin or a protein which provides a means of detection of the antibody or fragment thereof. Fusion proteins are constructed using recombinant DNA techniques known in the art (Sambrook et al. (1989), supra).

By “sample” is meant any sample comprising γ-H2AX histone proteins derived from or complexed with DNA, such as in the form of chromatin or reconstituted chromatin. By “derived from” is meant released from DNA, such as chromatin, as a result of natural or unnatural causes. A sample can comprise, for example, a protein extract, such as that used in Western blots or immunoblots. A sample can also comprise whole cells that have been fixed in order to preserve protein structure. Cells may be fixed using, for example, formaldehyde, which preserves protein structure and location within the cells and kills the cells. Cells can then be treated to render the cell membranes permeable to the anti-γ-H2AX antibody. The sample can be generated in a laboratory using routine methods or can be derived from an organism. In this regard, cells can be isolated from any source, i.e., blood or tissue samples from an animal.

A sample is contacted with a sufficient amount of antibody or antigenically-reactive fragment thereof for an adequate length of time to allow binding of the antibody to the phosphorylated H2AX protein. As such, the particular quantity of antibody used in the present inventive method will depend on various factors, such as the size of the sample, the temperature of the reaction, and the affinity of the specific antibody for the antigen. Optimization of binding conditions can easily be determined by the ordinary skilled artisan using routine experimentation.

Preferably, the isolated and purified antibody or antigenically-reactive fragment thereof for use in the present inventive method is labeled with a means of facilitating detection of the binding of the antibody or antigenically-reactive fragment thereof to γ-H2AX. Means of facilitating detection include, but are not limited to, an enzyme, a radioactive isotope, a fluorescent molecule, biotin and the like. Alternatively, a labeled secondary antibody can be used to detect binding of the antibody or antigenically-reactive fragment thereof to the γ-H2AX histone protein, as discussed below.

Binding of the antibody to the γ-H2AX histone protein can be detected by any number of methods widely used in the art such as, for example, those described in U.S. Pat. No. 6,362,317. For instance, antibody binding can be detected using Western blot or immunoblot techniques (see, for example, Frederick et al., Current Protocols in Molecular Biology, Wiley Interscience, New York, N.Y. (1987) or an enzyme linked immunoabsorbant assay (ELISA). See for example Coligan et al. Current Protocols in Immunology, Wiley Inter-Science New York, N.Y. (1996). Briefly, the antibody of the present invention, the 1° antibody, is allowed to recognize γ-H2AX proteins in a sample. Any unbound antibodies are washed away and a labeled 2° antibody is added. The 2° antibody specifically binds to the 1° antibody, thereby identifying 1° antibody-antigen complexes. Any antibody label detectable by conventional means is suitable for the present inventive method. Such labels include, for example, enzymes, such as peroxidase or luceriferase, radioactive isotopes, fluorescent molecules, such as fluorescein or rhodamine, biotin and the like.

Alternatively, a sample of cells, such as cells taken from blood, tissue, etc., is fixed and γ-H2AX is assayed directly in the cells by microscopy or flow cytometry, as illustrated in Example 4 of U.S. Pat. No. 6,362,317 and described in Spector et al., Cells, A Laboratory Manual, Cold Spring Harbor Laboratory Press, Cold Spring Harbor, N.Y. (1998). Use of microscopy or flow cytometry permits the direct measurement of antibody-γ-H2AX and, therefore, the number of DNA double-stranded breaks in individual cells. Fluorescent microscopy also enables the determination of the position of a DNA double-stranded break in the nucleus. Measurements from many cells can be automatically tabulated using flow or laser scanning cytometry.

The present invention can be used to quantify the level of double-stranded breaks in DNA. It is believed that, in terms of detection by microscopy, each antibody-γ-H2AX foci represents a single double-stranded break in DNA. Therefore, the number of double-stranded breaks present can be counted by counting the foci. This technique requires the use of a high-powered microscope and fluorescently labeled foci. Alternatively, total fluorescence in a sample can be quantified using techniques known in the art, such as, for example, flow or laser cytometry. The fluorescence of a particular sample can be compared to the fluorescence generated by standards comprising a known level of DNA double-stranded breaks. The level of DNA double-stranded breaks in the sample can then be estimated. Similarly, the level of DNA double-stranded breaks can be quantitated from a Western blot or immunoblot using densitometric techniques. Such techniques are widely used in the art. The density of the band corresponding to antibody-γ-H2AX complexes on a Western blot can be compared to the density of bands from standards, corresponding samples with known number of breaks, and the number of DNA double-stranded breaks in the sample quantified.

The information of how much γ-H2AX is phosphorylated and thereby the number of DNA double-stranded breaks that have occurred in a cancer patient can be used to determine the amount of the HDAC inhibitor, preferably a compound of Formula (I) and (II), that has reached the target site and whether the tumor is responsive to the drug. The level of -γ-H2AX present can be determined by taking a blood or tissue sample from the patient, preferably from about 5 to about 30 minutes, more preferably from about 15-25 minutes after administration of the HDAC inhibitor. The extent of γ-H2AX formed can be determined by comparing the sample with standards exposed to a predetermined amount of the HDAC inhibitor. For example, a standard curve can be developed in which matched cells, or cells that are identical to those assayed, are exposed to varying, predetermined amounts of the HDAC inhibitor and antibody-γ-H2AX quantitated. It is then possible to determine the level of γ-H2AX in a sample by comparing the level of γ-H2AX of the sample with the standard curve. Such techniques are widely used in the art.

The sample can be obtained by any method. A sample is “fixed” in order to prevent degradation (see, for example, Cell Biology, (Julio Celis ed.), Academic Press, San Diego, Calif. (1998)). For example, tissue or blood samples that will be used as a source of protein for Western blots are frequently frozen after extraction from an organism and assayed at a later date. The actual proteins extracted from the blood or tissue sample can also be frozen. In some situations, the sample is fixed using formaldehyde, thereby preserving protein structure and location within the cell. By determining the actual level of γ-H2AX formed using the method as described above, it is possible to optimize the dosage of an HDAC inhibitor for a particular patient.

One of ordinary skill in the art will appreciate the need to standardize sample preparation in order to examine accurately apoptosis. Preferably, the sample is taken from a pre-determined quantity of cells in order to reduce variability within measurements. The present inventive method is not dependent on the number of cells in a sample, although the sample must comprise an adequate number of cells to provide enough histone proteins to bind to an isolated or purified anti-γ-H2AX antibody or antigenically-reactive fragment thereof and be detectable. A skilled artisan can determine an adequate sample size using routine methods. Similarly, when measuring the effectiveness of a drug to induce apoptosis, samples should be obtained and fixed at set time points after administration of the drug in order to assure uniformity in measurements.

Wherein the present inventive method is used as an indicator of apoptosis, the method further comprises assessing the extent of apoptosis of cells in the sample by comparing the level of DNA double-stranded breaks detected for the sample to a standard. The greater the signal due to antibody binding to γ-H2AX, the greater the incidence of apoptosis. The standard comprises a sample from the patient prior to the administration of a compound of Formula (I). Other methods that can be used to detect γ-H2AX are described in MacPhail, S. H. et al. (2003) Radiation Research 159:759-767; Stiff, T. et al. (2004) Cancer Research 64:2390-2396; and Banath, J. and P. L. Olive (2003) Cancer Research 63:4347-4350.

The production of anti-γ-H2AX antibody and the determination of the DNA ds breaks can be carried out as described in U.S. Pat. No. 6,362,317, Examples 1 and 4 respectively. Alternatively, anti-γ-H2AX antibody sold by Cell Signaling Technology, Inc., can be used. A person skilled in the art will recognize the level of cytokeratin-18 fragment aa 387-397 formed in a cancer patient upon administration of a compound of Formula (I) can be measured utilizing the procedures described above by using an anti-cytokeratin-18 fragment aa 387-397 antibody. Other methods that can be used to detect cytokeratin-18 fragment aa 387-397 are described in Carr, N. (2000) Arch. Pathol. Lab. Med. 124:1768-1772; Biven, K. et al. (2003) Apoptosis 8:263-268; and Kramer, G. et al. (2004) Cancer Research 64:1751-1756.

### Testing

The ability of the compounds of this invention to cause phosphorylation of the histone variant γ-H2AX is determined utilizing an in vitro assay using the assay described below.

### Administration and Pharmaceutical Compositions

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors.

Therapeutically effective amounts of an HDAC inhibitor, in particular a compound of Formula (I) or (II), may range from approximately 0.1-50 mg per kilogram body weight of the recipient per day; preferably about 0.5-20 mg/kg/day. Thus, for administration to a 70 kg person, the dosage range would most preferably be about 35 mg to 1.4 g per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral or parenteral using a convenient daily dosage regimen, which can be adjusted according to the degree of affliction. Oral compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, an HDAC inhibitor, preferably a compound of Formula (I) or (II), in combination with at least one pharmaceutically acceptable excipient. Acceptable excipients are non-toxic, aid administration, and do not adversely affect the therapeutic benefit of the HDAC inhibitor. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18^{th} ed., 1990).

The amount of the compound in a formulation can vary within the full range employed by those skilled in the art. Typically, the formulation will contain, on a weight percent (wt %) basis, from about 0.01-99.99 wt % of an HDAC inhibitor based on the total formulation, with the balance being one or more suitable pharmaceutical excipients. Preferably, the compound is present at a level of about 1-80 wt %. Representative pharmaceutical formulations containing an HDAC inhibitor are described below.

### EXAMPLES

The following preparations and examples are given to enable those skilled in the art to more clearly understand and to practice the present invention. They should not be considered as limiting the scope of the invention, but merely as being illustrative and representative thereof.

### SYNTHETIC EXAMPLES

### Example 1

### Synthesis of N-hydroxy-4-(2-benzenecarbonylamino-ethoxy)benzamide

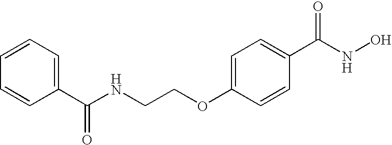

### Step 1

To a solution of 2-aminoethanol (3.1 g, 50 mmol) in THF (10 mL) was added tert-butyloxycarbonyl anhydride (10.9 g, 50 mmol) in THF (150 mL). The reaction mixture was stirred for 3 h, then diluted with ethyl acetate, washed with 0.5 M aqueous HCl, and brine. The organic layer was dried (MgSO₄), filtered and concentrated in vacuo to give 2-N-Boc-aminoethanol which was directly used in the next step.

### Step 2

To a solution of triphenylphosphine (17.7 g, 67.5 mmol) in anhydrous THF (135 mL) was added DIAD (13.6 g, 67.5 mmol). The solution was stirred until a white precipitate was formed (2 to 10 min). After additional 60 min., a solution of 2-N-Boc amino-ethanol (7.2 g, 45 mmol) and methyl 4-hydroxybenzoate (6.8 g, 45 mmol) in THF (25 mL) was added and stirring was continued for 5 h. The reaction mixture was concentrated in vacuo and purified by flash chromatography to give methyl 4-(2-N-Boc aminoethoxy)benzoate. Alternatively, the crude material can directly be used in the next step.

### Step 3

To a solution of crude methyl 4-(2-N-Boc aminoethoxy)benzoate in methanol (20 mL) was added 4M HCl/dioxane (180 mL). After stirring for 3 h, diethyl ether (300 mL) was added providing a white precipitate. The solid was collected, suspended in ethyl acetate and stirred for 15-20 min. The solid was collected again and dried under high vacuo providing methyl 4-(2-aminoethoxy)benzoate hydrochloride 6.3 g (60% over 2 steps).

### Step 4

To a suspension of methyl 4-(2-amino-ethoxy)benzoate hydrochloride (0.232 g, 1 mmol) in THF (6 mL) was added benzoyl chloride (0.140 g, 1 mmol) followed by triethylamine (0.121 g, 1.2 mmol). The reaction mixture was stirred for 1 h and then diluted with ethyl acetate. The organic layer was washed with 0.5 M aqueous HCl, saturated sodium bicarbonate solution, and brine. The organic layer was concentrated in vacuo to give methyl 4-(2-benzenecarbonylamino-ethoxy)benzoate which was directly used in the next step.

### Step 5

To a solution of crude methyl 4-(2-benzenecarbonylamino-ethoxy)benzoate (0.5 mmol) in a 1:1 mixture of THF/methanol (20 mL) was added 50 wt. % aqueous hydroxylamine (3 mL) followed by 1M aqueous NaOH (1 mL) adjusting the pH between 10-11. The reaction mixture was stirred for 14 h, neutralized to pH=7-8 with 6 M aqueous HCl and concentrated in vacuo. The precipitate was collected and purified by HPLC providing the title compound as a white solid. ¹H NMR (DMSO-d₆): δ 8.69 (t, J=5.8 Hz, 1H), 7.83 (d, J=7.5 Hz, 2H), 7.69 (d, J=9.1 Hz, 2H), 7.46 (m, 3H), 6.99 (d, J=9.1 Hz, 2H), 4.16 (t, J=5.8 Hz, 2H), 3.63 (q, J=5.8 Hz, 2H). EM (calc.): 300.1; MS (ESI) m/e: 301.1 (M−1)⁺, 299.0 (M+1)⁻.

Proceeding as described in Example 1, Steps 1-4 above, but substituting 2-aminoethanol with (S)-(+)-2-amino-1-butanol provided methyl 4-(2S-aminobutoxy)benzoate hydrochloride.

### Example 2

### Synthesis of N-hydroxy-4-[2-(benzofuran-2-yl-carbonylamino)-ethoxy]-benzamide

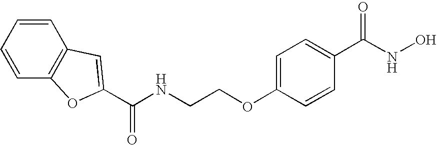

### Step 1

A mixture of benzofuran-2-carboxylic acid (0.162 g, 1 mmol), EDC.HCl (0.268 g, 1.4 mmol) and HOBT.H₂O (0.203 g, 1.5 mmol) in DMF (6 mL) was stirred for 2 h. Methyl 4-(2-aminoethoxy)benzoate hydrochloride (0.232 g, 1 mmol) was added followed by triethylamine (0.121 g, 1.2 mmol). The reaction mixture was stirred for 2 h and then diluted with ethyl acetate, washed with saturated sodium bicarbonate solution, and brine. The organic layer was concentrated in vacuo and the crude 4-[2-(benzofuran-2-ylcarbonylamino)ethoxy]benzoate was converted to the title compound as described in Example 1, Step 5 above. ¹H NMR (DMSO-d₆) δ 11.05 (s, 1H), 8.92 (t, J=5.6 Hz, 1H), 8.88 (s, 1H), 7.76 (d, J=8.0 Hz, 1H), 7.70 (d, J=9.2 Hz, 2H), 7.64 (d, J=8.0 Hz, 1H), 7.55 (s, 1H), 7.46 (t, J=6.8 Hz, 1H), 7.32 (t, J=8.0 Hz, 1H), 7.01 (d, J=8.2 Hz, 2H), 4.18 (t, J=5.6 Hz, 2H), 3.67 (m, 2H). EM (calc.): 340.1; MS (ESI) m/e: (M+1H)⁺: 341.0, (M−1H)⁻: 339.1.

### Example 3

### Synthesis of N-hydroxy-4-[2-(benzothiophen-2-yl-carbonylamino)-ethoxy]-benzamide

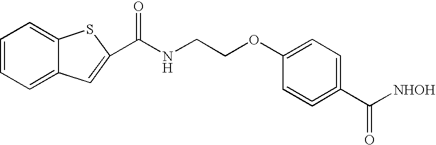

To a suspension of methyl 4-(2-aminoethoxy)benzoate hydrochloride (0.232 g, 1 mmol) in THF (6 mL) was added benzothiophene-2-carbonyl chloride (0.150 g, 1 mmol) followed by triethylamine (0.121 g, 1.2 mmol). The reaction mixture was stirred for 1 h and diluted with ethyl acetate (50 mL). The organic layer was washed with 0.5 N aqueous HCl, saturated sodium bicarbonate solution, and brine. The organic layer was concentrated in vacuo and the crude methyl 4-[2-(benzothiophen-2-yl-carbonylamino)ethoxy]benzoate was converted to the title compound as described in Example 1, Step 5 above.

Proceeding as described in Example 3 above, but substituting methyl 4-(2-aminoethoxy)benzoate hydrochloride with methyl 4-(2S-aminobutoxy)benzoate hydrochloride and benzothiophene-2-carbonyl chloride with cinnamoyl chloride provided N-hydroxy-4-[2S-(trans-cinnamoylamino)butoxy]benzamide.

### Example 4

### Synthesis of N-hydroxy-4-[2-(3-dimethylaminobenzofuran-2-ylcarbonylamino)-ethoxy]-benzamide

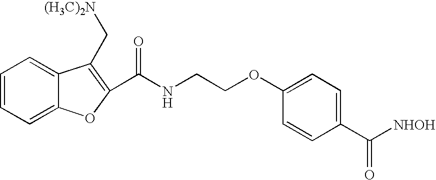

To a solution of 3-methyl-benzofuran-2-carboxylic acid (0.98 g, 5.6 mmol) and 5 drops of DMF in THF (25 mL) was added oxalyl chloride (0.53 mL, 6.1 mmol). After stirring the solution for 1 h at room temperature, methanol (20 mL) and TEA (7 mL) were added. The slurry was stirred overnight at room temperature, then concentrated and dissolved in ethyl acetate (100 mL) and washed with mild NaHCO₃ (100 mL). The organic layer was dried (MgSO₄), filtered and concentrated to collect 3-methylbenzofuran-2-carboxylic acid methyl ester (1 g) as a tan solid. The crude methyl ester was used without further purification.

### Step 2

A solution of 3-methylbenzofuran-2-carboxylic acid methyl ester (1.0 g, 5.3 mmol), NBS (0.95 g, 5.3 mmol) and AIBN (87 mg, 0.53 mmol) was heat to reflux in CCl₄ (40 mL) for 3 h, then cooled to room temperature and concentrated. The residue was dissolved in ethyl acetate (100 mL) and washed with water (100 mL). The organic layer was dried (MgSO₄), filtered and concentrated to collect 3-bromomethylbenzofuran-2-carboxylic acid methyl ester (1.55 g) of a tan/yellow solid which was used in the next step without further purification.

### Step 3

3-Bromomethylbenzofuran-2-carboxylic acid methyl ester (269 mg, 1 mmol) was dissolved in anhydrous DMF and added 2M dimethylamine/THF solution (1.5 mL, 3 mmol). After 1-2 h, the reaction was diluted with EtOAc and washed twice with saturated NaHCO₃ (aq.) and brine. The organic extract over was dried over Na₂SO₄ and then concentrated in vacuo. The crude was purified on a silica gel column (5% MeOH in dichloromethane) to give 3-dimethylaminomethylbenzofuran-2-carboxylic acid methyl ester (131 mg).

### Step 4

To a solution of 3-dimethylaminomethylbenzofuran-2-carboxylic acid methyl ester (131 mg, 0.56 mmol) in MeOH was added 1M NaOH(aq.) till the pH of the solution was 13. The reaction mixture was stirred for 60-90 min. Upon completion, the reaction mixture was acidified to pH 3 with HCl (aq.) and concentrated to dryness to give 3-dimethylaminomethylbenzofuran-2-carboxylic acid as the HCl salt, which was used for next step without further purification.

### Step 5

To 3-dimethylaminomethylbenzofuran-2-carboxylic acid (140 mg, 0.56 mmol) was added EDC.HCl (150 mg, 0.784 mmol) and HOBt-H₂O (114 mg, 0.84 mmol) in anhydrous DMF. The reaction mixture was stirred for 30-60 mins, after which methyl-(4-(2-ethoxyamine))benzoate hydrochloric salt (130 mg, 0.56 mmol) and triethylamine (94 μL, 0.672 mmol) were added and the reaction was stirred overnight. The reaction mixture was diluted with EtOAc and washed twice with saturated NaHCO₃ (aq.) and brine. The organic extract was concentrated in vacuo to give methyl 4-[2-(3-dimethylaminobenzofuran-2-yl-carbonylamino)ethoxy]benzoate, which was then used without further purification.

### Step 6

To a solution of crude methyl 4-[2-(3-dimethylaminobenzofuran-2-yl-carbonylamino)-ethoxy]-benzoate in MeOH and THF was added excess aqueous hydroxylamine solution and NaOH (aq.) to give pH 10-11. The reaction mixture was stirred overnight and then neutralized to pH 7-8 with aqueous hydrochloric acid and concentrated in vacuo. The residue was dissolved in acetonitrile and water and purified with prep HPLC to give the title compound (107 mg).

¹HNMR (400 MHz, DMSO-d₆) δ 9.88 (m, 1H), 9.31 (t, J=6.0 Hz, 1H), 8.04 (d, J=7.6 Hz, 1H), 7.70 (m, 3H), 7.57 (t, J=7.6 Hz, 1H), 7.45 (t, J=7.6 Hz, 1H), 6.99 (d, J=9.2 Hz, 2H), 4.76 (d, J=4.8 Hz, 2H), 4.23 (t, J=6.0 Hz, 2H), 3.71 (m, 2H), 2.84 (s, 3H), 2.83 (s, 3H). EM (calc.): 397.2; MS (ESI) m/e (M+1H): 398.1, (M−1H): 396.2.

### Example 5

### Synthesis of N-hydroxy-4-{2-[3-(2,2,2-trifluoroethyloxymethyl)benzofuran-2-yl-carbonylamino]ethoxy}benzamide

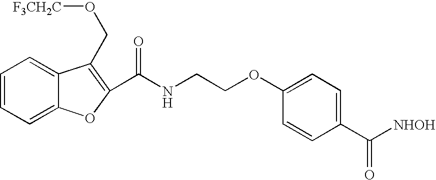

### Step 1

Sodium hydride (15 mg, 0.56 mmol) was suspended in anhydrous DMF and stirred under N₂(g). 2,2,2-Trifluoroethanol (270 μL, 3.7 mmol) was added and after stirring the reaction mixture for 15-20 min., 3-bromomethylbenzofuran-2-carboxylic acid methyl ester was added. After 8 h, 1M NaOH (aq.) was added and the reaction mixture was stirred for 10-15 mins. The reaction mixture was acidified reaction to pH 3 with aqueous hydrochloric acid and the product was extracted with EtOAc. The organic layer was dried organic over Na₂SO₄ and concentrated in vacuo to give 3-(2,2,2-trifluoroethoxymethyl)benzofuran-2-carboxylic acid (38 mg) which was then used without purification.

### Step 2

To a solution of 3-(2,2,2-trifluoroethoxymethyl)benzofuran-2-carboxylic acid (38 mg, 0.139 mmol) in anhydrous DMF was added EDC.HCl (37 mg, 0.195 mmol) and HOBt-H₂O (26 mg, 0.195 mmol). After 60-90 mins, methyl-(4-(2-ethoxyamine))benzoate hydrochloric salt (32 mg, 0.139 mmol) and triethylamine (23 μL, 0.167 mmol) were added and the reaction mixture was stirred for 1-2 h. The reaction mixture was diluted with EtOAc and washed twice with saturated NaHCO₃(aq.) and the organic extract was concentrated to give methyl 4-{2-[3-(2,2,2-trifluoroethoxymethyl)benzofuran-2-yl-carbonylamino]ethoxy}benzoate, which was then used without further purification.

### Step 3

4-{2-[3-(2,2,2-Trifluoroethoxymethyl)benzofuran-2-yl-carbonylamino]-ethoxy}-benzoate was dissolved in MeOH and excess aqueous hydroxylamine solution and NaOH(aq) were added to give pH 10-11. After stirring overnight, the reaction mixture was neutralized reaction to pH 7-8 with aqueous hydrochloric acid. The reaction mixture was concentrated in vacuo to give a solid which was collected and washed with water, then dissolved in acetonitrile and water and purified with prep HPLC to give the title compound (35 mg).

¹H NMR (400 MHz, DMSO-d₆) δ 11.04 (s, 1H), 8.95 (t, J=5.6 Hz, 1H), 8.89 (s, 1H), 7.81 (d, J=7.6 Hz, 1H), 7.70 (d, J=8.8 Hz, 2H), 7.63 (d, J=8.8 Hz, 1H), 7.50 (t, J=8.8 Hz, 1H), 7.36 (t, J=8.0 Hz, 1H), 7.00 (d, J=9.2 Hz, 2H), 5.25 (s, 2H), 4.18 (m, 4H), 3.67 (m, 2H) EM (calc.): 452.1; MS (ESI) m/e (M+1H)⁺: 453.0, (M−1H)⁻: 451.0.

### Example 6

### Synthesis of N-hydroxy-4-{2-[5-(2-pyrrolidin-1-ylethyloxy)benzofuran-2-ylcarbonylamino]-ethoxy}benzamide

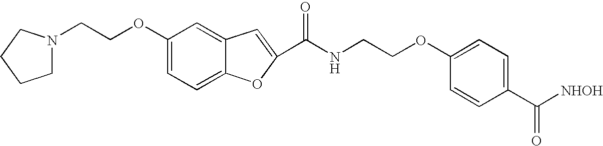

### Step 1

5-Methoxybenzofuran-2-carboxylic acid (5.04 g, 26 mmol) was weighed into a 200 mL round bottom flask fitted with a stir bar, septum and nitrogen inlet. Anhydrous MeOH (50 mL) was added under nitrogen atmosphere. The solution was cooled in an ice bath and thionyl chloride (2.3 mL, 32 mmol) was added dropwise with vigorous stirring. After stirring for 72 h at room temperature, the reaction mixture was poured into water (150 mL) and the white solid was collected. The solid was dissolved in toluene (100 mL) and the solution was washed with 1M NaHCO₃ and brine and dried over MgSO₄. Removal of the organic layer provided 5-methoxybenofuran-2-carboxylic acid methyl ester as a white solid (5.15 g).

### Step 2

A solution of 5-methoxybenzofuran-2-carboxylic acid methyl ester (5.15 g, 25 mmol) in anhydrous methylene chloride (15 mL) was cooled to 40° C. under nitrogen atmosphere. Boron tribromide in CH₂Cl₂ (27 mL of 1.0 M) was added over 1 h using a syringe pump. The reaction mixture was allowed to warm to room temperature. After 16 h, the reaction mixture was cooled in an ice bath and quenched with MeOH (15 mL). The reaction mixture was poured into brine (100 mL) and extracted with EtOAc. The organic extracts were dried over anhydrous MgSO₄, and the solvent was removed on rotary evaporator. The residue was triturated with hexane and the yellow solid was filtered and dissolved in anhydrous MeOH (30 mL). The solution was cooled in an ice bath and thionyl chloride (1.9 mL, 26 mmol) was added dropwise. After 72 h, water (100 mL) was added and solid was collected. Purification of the crude product on a 300 cm³ silica gel in a 5×15 cm plug using EtOAc provided 5-hydroxy-benzofuran-2-carboxylic acid methyl ester (4.53 g).

### Step 3

Anhydrous tetrahydrofuran (15 mL) was added to a mixture of 5-hydroxybenzofuran-2-carboxylic acid methyl ester (1.10 g, 5.7 mmol), triphenylphosphine (1.50 g, 5.7 mmol), and 1-(2-hydroxyethyl)-pyrrolidine (0.66 g, 5.7 mmol) under a nitrogen atmosphere. Diisopropyl azodicarboxylate (1.15 mL, 5.8 mmol) was slowly added to the solution at room temperature. After 2 days, the solvent was removed and the residue was dissolved in a 2:1 mixture of Et₂O:EtOAc (150 mL). The solution was washed with 1.0M aqueous NaOH. The product was extracted into 1.0 N HCl and the combined acid extracts were washed with Et₂O. The extracts were cooled and the pH of the extracts was adjusted to pH 12 with 50% aqueous NaOH. The basic solution was extracted with CH₂Cl₂ and the organic layer was dried over anhydrous MgSO₄, and concentrated to give 5-(2-pyrrolidin-1-yl-ethoxy)benzofuran-2-carboxylic acid methyl ester (0.96 g) as an amber colored solid.

### Step 4

To an ice-cooled solution of 5-(2-pyrrolidin-1-ylethoxy)benzofuran-2-carboxylic acid methyl ester (960 mg, 3.3 mmol) anhydrous ethylene glycol dimethyl ether (10 mL) was added dropwise degassed aqueous lithium hydroxide solution (2.0 mL, 2.0M). After stirring at room temperature for 4 h, the solution was cooled down and the pH was adjusted to 2 with 4.0 N HCl in dioxane. A gummy tan precipitate formed. The solvent was removed and the gummy residue was frozen and lyophilized. The tan colored solid was dissolved in boiling 2-propanol (90 mL), the solution was filtered hot and then cooled to give 5-(2-pyrrolidin-1-yl-ethoxy)-benzofuran-2-carboxylic acid as beige colored needles (528 mg). Additional 153 mg was obtained from the mother liquor.

### Step 5

To a solution of 5-(2-pyrrolidin-1-ylethoxy)benzofuran-2-carboxylic acid (156 mg, 0.50 mmol) and 4-(2-aminoethoxy)benzoic acid methyl ester hydrochloride (129 mg, 0.56 mmol) in DMF (4.5 mL) in a 20 mL vial was added diisopropylethylamine (0.88 mL, 5.1 mmol). A solution of O-(7-azabenzotriazol-1-yl)-1,1,3,3-tetramethyluronium hexafluorophosphate (740 μL, 0.82 M, 0.61 mmol) in DMF was added to give a bright yellow solution. The vial was purged with nitrogen and stirred at room temperature for 18 h. The solution was removed and the residue was dissolved in EtOAc (25 mL) and washed with H₂O, 1.0M aqueous K₂CO₃, and brine. The organic layer was dried over anhydrous MgSO₄ and solvent was removed on a rotary evaporator. Purification of the residue by column chromatography on a silica gel column using 93:5:2 CH₂Cl₂:MeOH:TEA eluent provided 4-{2-[5-(2-pyrrolidin-1-ylethoxy)benzofuran-2-carbonylamino]-ethoxy}-benzoic acid methyl ester as a beige solid (174 mg).

### Step 6

To a solution of 4-{2-[5-(2-pyrrolidin-1-ylethoxy)benzofuran-2-carbonylamino]-ethoxy}-benzoic acid methyl ester (169 mg, 0.37 mmol) in methanol (8 mL) and tetrahydrofuran (4 mL) was added hydroxylamine in water (2.9 mL of a 50 wt. % solution) and 4.0 M aqueous solution of sodium hydroxide (0.65 mL). After stirring for 18 h, the organics were removed and the aqueous solution was cooled in an ice/water bath and the pH was adjusted to ˜8 with 4.4 mL 1.0 N HCl to give precipitates. The heterogeneous solution was warmed to room temperature and acetonitrile was added till the precipitates dissolved. The solution was chromatographed on C-18 reverse phase HPLC. Fractions with absorbance at 214 nm, were collected, frozen, and lyophilize to give the title compound (31 mg). ¹H NMR (400 MHz, DMSO-d₆) δ: 11.05 (s, 1H), 10.4 (s, 1H), 8.91 (s, 2H), 7.70 (d, 2H, J=7.4), 7.59 (dd, 1H, J=3.7, 9.1 Hz), 7.51 (d, 1H, J=3.7 Hz), 7.35 (s, 1H), 7.13 (d, 1H, J=9.0 Hz), 7.00 (d, 2H, J=7.4 Hz), 4.37 (m, 2H), 4.18 (m, 2H), 3.62 (m, 6H), 3.12 (m, 2H), 2.02 (m, 2H), 1.89 (m, 2H). EM (calc.): 453.2; MS (ESI) m/e (M+1H)⁺: 454.1, (M−1H)⁻: 452.2.

### Example 7

### Synthesis of N-hydroxy-4-[2-(3-dimethylaminobenzofuran-2-ylcarbonylamino)-ethoxy]-benzamide hydrochloride

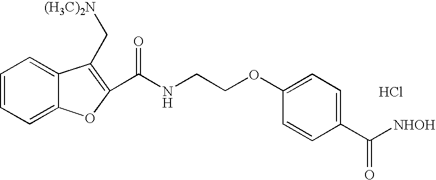

### Step 1

(2-Hydroxyethyl)carbamic acid tert-butyl ester (152.0 g, 0.942 mol) and 4-hydroxy-benzoic acid methyl ester (174.0 g, 1.12 mol) were dissolved in tetrahydrofuran (2000 mL) and cooled to 0-5° C. Triphenylphosphine (292.8 g 1.116 mol) was added to the cooled mixture. A solution of diisopropyl azodicarboxylate (246.0 g, 1.218 mol) in tetrahydrofuran (400 mL) was added dropwise over a period of one to two hours keeping the reaction temperature below 10° C. After addition, the reaction was allowed to warm slowly to ambient temperature and stirred overnight. After completion of reaction, solvent was distilled under reduced pressure and the resulting oil was dissolved in ethanol (500 mL) and ethyl acetate (2 L). Acetyl Chloride (222.0 g, 2.826 mol) was added drop wise over fifteen minutes with the temperature allowed to rise to 40° C. The resulting suspension was stirred at 40° C. until completion of reaction. After completion of reaction, the resulting crystals were filtered on a coarse frit and washed with ethyl Acetate (300 mL). The material is dried in vacuo to give of 4-(2-aminoethoxy)benzoic acid methyl ester hydrochloride (204.1 g) as a white crystalline solid.

### Step 2

4-(2-Aminoethoxy)benzoic acid methyl ester hydrochloride (78.90 g, 0.340 mol) and 3-methylbenzofuran-2-carboxylic acid (60.0 g, 0.340 mol) were suspended in acetonitrile (360 mL) and cooled to 0-5° C. Pyridine (137.6 mL, 1.702 mol) was added quickly. A solution of phosphorous oxychloride (52.2 g, 0.340 mol) in acetonitrile (60 mL) was added drop wise over thirty to forty-five minutes with the temperature kept below 20° C. The reaction mixture was allowed to stir for one hour and warm slowly to ambient temperature. After completion of reaction, the solution was added to a rapidly stirred 0-5° C. mixture of chlorobenzene (1 L) and 1N HCl (1 L). The reaction mixture was stirred rapidly and allowed to warm to room temperature. The organic layer was washed with water, 3% potassium hydroxide, and again with water. Chlorobenzene (100 mL) was added to the washed organic layer. Solvent (100 mL) was then distilled at atmospheric pressure until the pot temperature reached 132° C. After cooling to ambient temperature, 4-{2-[(3-methylbenzofuran-2-carbonyl)amino]ethoxy}-benzoic acid methyl ester was stored in solution for use in the next step.

### Step 3

A solution of 4-{2-[(3-methylbenzofuran-2-carbonyl)amino]ethoxy}benzoic acid methyl ester (0.340 mol) in chlorobenzene (1 L) was treated with 2,2′-azobisisobutyronitrile (5.60 g, 0.017 mol) and N-bromosuccinimide (75.76 g, 0.426 mol). The resulting mixture is heated to 80° C. and stirred for one hour. After completion of reaction, the reaction mixture was cooled to ambient temperature and washed with water, 3% sodium hydrogensulfite, and again with water. Solvent was distilled under reduced pressure and after cooling to ambient temperature, dichloromethane was added and to give 4-{2-[(3-bromo-methylbenzofuran-2-carbonyl)amino]-ethoxy}benzoic acid methyl ester which was used in the next step.

### Step 4

A solution of 4-{2-[(3-bromomethylbenzofuran-2-carbonyl)amino]ethoxy}benzoic acid methyl ester (0.340 mol) in chlorobenzene (200 mL) and dichloromethane (800 mL) was added dropwise to a 0-5° C. solution of 2M dimethylamine in tetrahydrofuran (510 mL, 1.022 mol) over 30 minutes with the temperature below 20° C. The resulting mixture was stirred for one hour and allowed to warm to ambient temperature. After completion of reaction, the reaction mixture was washed with 5% potassium carbonate and water. Solvent was distilled at atmospheric pressure until the pot temperature reached 100° C. After cooling to 50° C., acetonitrile (400 mL) and ethyl Acetate (400 mL) were added to the pot. The reaction mixture was heated to reflux until all solids dissolved. The reaction mixture was allowed to cool to give 4-{2-[(3-dimethylaminomethyl-benzofuran-2-carbonyl)amino]ethoxy}benzoic acid methyl ester (76.6 g) as an off white powder.

### Step 5

4-{2-[(3-Dimethylaminomethylbenzofuran-2-carbonyl)amino]ethoxy}benzoic acid methyl ester (70.0 g, 0.177 mol) was suspended in methanol (350 mL). 50% Potassium hydroxide (139.8 g, 1.062 mol) was added and the reaction mixture was heated to 60° C. until completion of reaction. After cooling to room temperature, the resulting crystals were filtered on a coarse frit and washed with methanol. The crystals were dried in vacuo to give 4-{2-[(3-dimethylaminomethylbenzofuran-2-carbonyl)amino]ethoxy}benzoic acid potassium salt (72.0 g) as a white solid.

### Step 6

4-{2-[(3-Dimethylaminomethylbenzofuran-2-carbonyl)amino]ethoxy}benzoic acid potassium salt (20.0 g, 0.0476 mol) was suspended in N,N-dimethylformamide (100 mL). 4 Molar hydrochloric acid in dioxane (11.9 mL, 0.0476 mol) was added to the suspension. After stirring for thirty minutes at ambient temperature, the reaction mixture was filtered through a medium frit. 1-[3-(Dimethylamino)propyl]-3-ethylcarbodiimide hydrochloride (10.94 g, 0.0571 mol) and 1-hydroxybenzotriazole (7.71 g, 0.0571 mol) were added to the solution and the reaction mixture was stirred one hour at ambient temperature. In a separate pot, hydroxylamine hydrochloride (13.2 g, 1.904 mol) was suspended in N,N-dimethylformamide (100 mL) and treated with triethylamine (33.1 mL, 2.380 mol). After stirring the reaction mixture for 1 h, the salt was filtered off and the resulting solution was added to the above activated acid solution and stirred at ambient temperature until completion of reaction. After the product began to crystallize, methanol (150 mL) was added slowly over thirty min. The reaction mixture was stirred for 1 h at ambient temperature then cooled to 0-5° C. and stirred another hour. The crystals were filtered and washed with methanol (40 mL) before being dried in vacuo to give 3-dimethylaminomethylbenzofuran-2-carboxylic acid [2-(4-hydroxy-carbamoylphenoxy)ethyl]amide (11.88 g) as a white solid. The crude material (13.25 g, 0.033 mol) was suspended in N,N-dimethylformamide (80 mL) and heated to 100° C. to give a solution. After cooling, ethanol (80 mL) was added drop-wise over thirty minutes and the resulting suspension was allowed to cool for an hour. The crystals were filtered and washed with ethanol (40 mL) and dried to give pure product (9.82 g) as a white solid.

### Step 7

3-Dimethylaminomethylbenzofuran-2-carboxylic acid [2-(4-hydroxy-carbamoylphenoxy)-ethyl]amide (22.7 g, 0.057 mol) was suspended in 2-propanol (220 mL). 12 N HCl (5.2 mL, 0.063 mol) was added in one portion and the resulting mixture was heated to reflux. Water (44 mL) was added drop-wise until an homogenous solution was obtained. The reaction mixture was allowed to cool and crystallize overnight. After cooling below 5° C. for one h, the crystals were filtered and washed with 2-propanol before being dried in vacuo to give the title compound (22.0 g) as a white solid.

### Example 8

### Synthesis of N-hydroxy-4-[2-(benzofuran-2-ylcarbonylamino)-ethylsulfanyl]benzamide

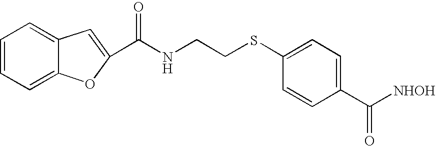

### Step 1

To a solution of diisopropyl azodicarboxylate (DIAD, 4.04 g, 20 mmol) in THF (100 mL) at 0° C. was added triphenylphosphine (5.25 g, 20 mmol). After 1 h, a solution of Boc-ethanolamine (3.22 g, 20 mmol) in THF (10 mL) was added. After 20 min, a solution of methyl 4-mercaptobenzoate (3.86 g, 20 mmol) in THF (10 mL) was added and the reaction mixture was stirred overnight at room temperature. The reaction mixture was concentrated and ethyl acetate (150 mL) was added. The solution was washed with 1N HCl, saturated aqueous NaHCO₃, brine, dried over MgSO₄, filtered, and evaporated to dryness. The oily yellow residue was eluted through a plug of silica gel (0-20 ethyl acetate in hexane as mobile phase) and the product was then recrystallized from ether and hexane to give methyl 4-(2-tert-butoxycarbonylaminoethylsulfanyl)benzoate (4.00 g).

### Step 2

A solution of methyl 4-(2-tert-butoxycarbonylaminoethylsulfanyl)benzoate (1.00 g, 3.21 mmol) in dichloromethane (8 mL) was treated with a solution of HCl in dioxane (4N, 8 mL, 10 equiv.) at room temperature for 3 h. Ether (100 mL) was added and the mixture was filtered, washed with ether and pumped dry to give methyl 4-(2-aminoethylsulfanyl)benzoate hydrochloride.

### Step 3

Methyl 4-(2-aminoethylsulfanyl)benzoate hydrochloride (0.248 g, 1.00 mmol), was combined with benzofuran-2-carboxylic acid (0.162 g, 1.00 mmol) and HBTU (0.379 g, 1.00 mmol) in DMF (5 mL) at room temperature. Triethylamine (0.307 mL, 2.2 mmol) was added and the reaction mixture was stirred at room temperature overnight. Saturated aqueous NaHCO₃ (15 mL) was added to give precipitates which was broken up by the addition of water (20 mL). The solid was filtered and the cake was dissolved in ethyl acetate. The residual water was removed by pipette and hexane was added to give methyl 4-{2-[(benzofuran-2-ylcarbonyl)amino]ethyl-sulfanyl}benzoate (0.138 g) as a gum which was used in the next step without further purification.

### Step 4

To a solution of methyl 4-{2-[(benzofuran-2-yl-carbonyl)amino]ethylsulfanyl}benzoate in THF (2 mL) was added a solution of 50% hydroxylamine in water (4 mL). Methanol (2 mL) and 0.1 M NaOH (0.11 mL) were added. The reaction mixture was stirred for three days at room temperature. The solvents were evaporated and the residue was crystallized from dichloromethane/ethyl acetate, to give title compound (46 mg). ¹H NMR (DMSO-d₆): 8.99 (1H, br. s), 8.96 (1H, t), 7.78 (1H, d), 7.7 (2H, d), 7.62 (1H, d), 7.53 (1H, s), 7.45 (1H, m*), 7.42 (2H, d), 7.33 (1H, t), 3.5 (2H, m), 3.12 (2H, m); MS (M+1)⁺: 357.

### Example 9

### Synthesis of N-hydroxy-4-[2-(benzofuran-2-ylcarbonylamino)ethylsulfonyl]benzamide

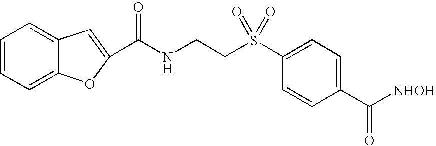

### Step 1

To a solution of methyl 4-(2-tert-butoxycarbonylaminoethylsulfanyl)-benzoate (3.00 g, 9.63 mmol) in methanol/water (1:1, 100 mL) was added Oxone® (13.03 g, 21.19 mmol). After 48 h, methanol was removed under reduced pressure, and the residue was partitioned between ethyl acetate (150 mL) and saturated aqueous NaHCO₃ (150 mL). The organic phase was washed with brine (100 mL), dried over MgSO₄, filtered, concentrated in vacuo, and the residue was recrystallized from ethyl acetate/hexane to give methyl 4-(2-tert-butoxycarbonylamino-ethylsulfonyl)benzoate (2.86 g) of the product.

### Step 2

A solution of methyl 4-(2-tert-butoxycarbonylaminoethylsulfonyl)benzoate (2.86 g, 8.33 mmol) in dichloromethane (20 mL) was treated with 4N HCl in dioxane (20 mL) for 2 h. Ether (200 mL) was added and the suspension was filtered, washed with ether (2×50 mL), hexane (50 mL) and pumped dry to give methyl 4-(2-aminoethylsulfonyl)benzoate hydrochloride (2.23 g) which was coupled with benzofuran 2-carboxylic acid as described above to afford the title compound. MS (M+1)⁺: 388.

### Example 10

### N-hydroxy-4-{3-[5-(N,N-dimethylaminoethoxy)indol-2-ylcarbonyl-amino]-prop-1-ynyl}-benzamide hydrochloride

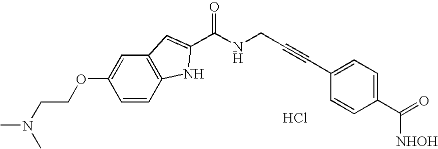

Example 10 was prepared as described in WO 05/019174. ¹H NMR (400 MHz, DMSO-d₆) δ 11.59 (s, 1H), 11.29 (s, 1H), 9.85 (s, 1H), 9.10 (s, 1H), 9.05 (t, J=5.5 Hz, 1H), 7.74 (d, J=8.3 Hz, 2H), 7.51 (d, J=8.3 Hz, 2H), 7.37 (d, J=8.8 Hz, 1H), 7.21 (d, J=2.4 Hz, 1H), 7.13 (d, J=1.7 Hz, 1H), 6.93 (dd, J₁=2.4 Hz, J₂=8.9 Hz, 1H), 4.40 (d, J=5.5 Hz, 2H), 4.33 (t, J=4.8 Hz, 2H), 3.52 (t, J=4.8 Hz, 2H), 2.88 (s, 6H); EM (calc.): 420.2; MS (ESI) m/e (M+1H)⁺: 421.0, (M−1H)⁻: 419.2.

### Example 11

### Synthesis of N-hydroxy-4-[3-(benzothiophen-2-ylcarbonylamino)prop-1-ynyl]-benzamide

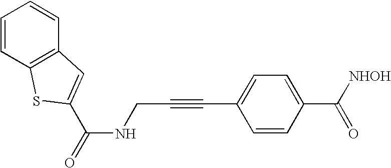

### Step 1

To a solution of propargylamine (5.50 g, 100 mmol) in THF (50 mL) was added tert-butyloxycarbonyl anhydride (21.8 g, 100 mmol) in THF (50 mL) and triethylamine (16.7 mL, 120 mmol). The reaction mixture was stirred for 3 h, then diluted with ethyl acetate (200 ml), washed with 0.5 N aqueous HCl (150 mL), and finally with brine (150 mL). The organic layer was dried over sodium sulfate, filtered, and concentrated in vacuo. The crude N-Boc-propargylamine was directly used in the next step without further purification.

### Step 2

To a solution of N-Boc-propargylamine (2.07 g, 13.4 mmol), methyl 4-iodobenzoate (3.50 g, 13.4 mmol) and PdCl₂(PPh₃)₂ (0.938 g, 1.34 mmol) in DMF (20 mL) was added triethylamine (9.31 ml, 126 mmol). The reaction mixture was stirred for 30 min at room temperature. Cu^{(I)}I (0.508 g, 2.67 mmol) was added and stirring was continued for additional 16 h. The reaction mixture was diluted with ethyl acetate (250 mL), washed with 0.5 N aqueous HCl (200 mL), and finally with brine (200 ml). The organic layer was dried over sodium sulfate, filtered, and concentrated in vacuo. Purification by flash chromatography over silica gel with ethyl acetate/hexane (1/4) provided methyl 4-(N-Boc-3-aminoprop-1-ynyl)benzoate (3.44 g) as highly viscous oil.

### Step 3

To a solution of methyl 4-(N-Boc-3-aminoprop-1-ynyl)benzoate (3.40 g, 11.7 mmol) in THF (25 mL) was added 4 N HCl/dioxane (25 ml, 100 mmol). The reaction mixture was stirred for 1 h. The formed precipitate was collected, washed with diethyl ether (200 mL) and dried in vacuo providing methyl 4-(3-aminoprop-1-ynyl)benzoate hydrochloride (2.46 g) as white solid. Alternatively, the N-Boc protected amine can be dissolved in methanol instead of THF. In this case the solvent was removed in vacuo and the precipitate was washed with diethyl ether.

### Step 4

To a suspension of methyl 4-(3-aminoprop-1-ynyl)-benzoate hydrochloride (0.226 g, 1 mmol) in THF (6 mL) was added benzothiophene-2-carbonyl chloride (0.150 g, 1.0 mmol) followed by triethylamine (0.253 g, 2.5 mmol). The reaction mixture was stirred for 1 h and diluted with ethyl acetate (50 mL). The organic layer was washed with 0.5 N aqueous HCl (50 mL), with saturated sodium bicarbonate solution (50 mL), and finally with brine. The organic layer was concentrated in vacuo and the crude material was directly used in the next step.

### Step 5

To a solution of methyl 4-[3-(benzothiophen-2-ylcarbonylamino)prop-1-ynyl]-benzoate (0.5 mmol) in THF/methanol (10 mL/10 mL) was added 50 wt. % aqueous hydroxylamine (3 mL) followed by 1M aqueous NaOH (1 mL) adjusting the pH to 10-11. The reaction mixture was stirred for ˜14 h, neutralized to pH=7-8 with 6 N aqueous HCl, and concentrated in vacuo. The precipitate was collected and purified by HPLC providing the title compound as a white solid.

### Example 12

### N-hydroxy-4-[3-(3-(4-chlorophenyl)-ureido)prop-1-ynyl]-benzamide

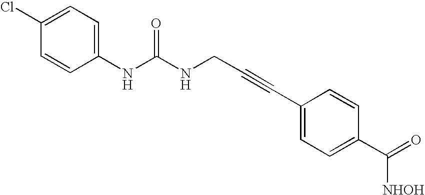

### Step 1

To a solution of methyl 4-(3-aminoprop-1-ynyl)benzoate hydrochloride (see Example 11, Steps 1-3 above; 150 mg, 0.66 mmol) in THF (5 mL) was added 4-chlorophenyl isocyanate (102 mg, 0.66 mmol) and triethylamine (278 μL, 2.0 mmol). The reaction mixture was stirred for 30 min, diluted with ethyl acetate (50 mL), and sequentially washed with water (25 mL), 0.5 N aqueous HCl (25 mL), saturated sodium bicarbonate (25 mL), and finally with brine (25 mL). The organic phase was dried over sodium sulfate and concentrated in vacuo to provide methyl 4-[3-(3-(4-chlorophenyl)-ureido)prop-1-ynyl]-benzoate as a white solid.

### Step 2

Methyl 4-[3-(3-(4-chlorophenyl)-ureido)prop-1-ynyl]-benzoate was converted to the title compound as described in Example 11, Step 5 above.

### Example 13

### Synthesis of N-hydroxy-4-[4-(1H-indol-2-yl-carbonylamino)piperidin-4-yl-ethynyl]-benzamide

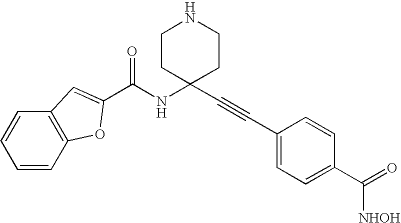

### Steps 1-6

1-N-Boc-4,4-amino-piperidinylcarboxylic acid was converted to methyl 4-(4-amino-piperidin-4-ylethynyl)-benzoate dihydrochloride salt as described in Example 11, Steps 1-6 above.

### Step 7

A solution of methyl 4-(4-aminopiperidin-4-ylethynyl)-benzoate dihydrochloride (791 mg, 2.4 mmol) in THF (15 mL) was treated with triethylamine (1.3 mL, 9.6 mmol) and di-tert-butyl dicarbonate (521 mg, 2.4 mmol). After 1 h, the reaction mixture was diluted with ethyl acetate (150 mL) and the organic layer was washed with water and brine, dried over sodium sulfate, and concentrated in vacuo to provide N-Boc-methyl 4-(4-amino-piperidin-4-ylethynyl)-benzoate (930 mg, 2.6 mmol) as yellowish foam which was used in the next step without further purification.

### Step 8

N-Boc methyl 4-(4-aminopiperidin-4-ylethynyl)-benzoate was converted to N-Boc-methyl 4-[4-(benzofuran-2-yl-carbonylamino)piperidin-4-ylethynyl]-benzoate as described in WO 05/019174.

### Step 9

N-Boc methyl 4-[4-(benzofuran-2-ylcarbonylamino)piperidin-4-ylethynyl]benzoate was converted to methyl 4-[4-(benzofuran-2-ylcarbonylamino)piperidin-4-ylethynyl]benzoate hydrochloride salt as described in Example 11, Step 3 above.

### Step 10

Methyl 4-[4-(benzofuran-2-ylcarbonylamino)piperidin-4-ylethynyl]benzoate hydrochloride was converted to the title compound as described in Example 11, Step 5 above.

### Example 14

### Synthesis of N-hydroxy-4-[4-(1H-indol-2-yl-carbonylamino)-1-(2,2,2-trifluoro-ethyl)-piperidin-4-yl-ethynyl]-benzamide

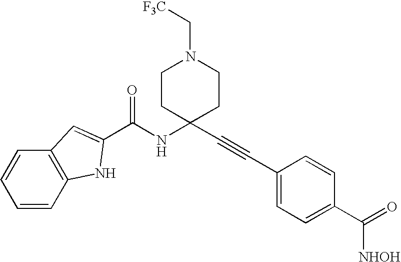

### Steps 1-9

Methyl 4-[4-(1H-indol-2-ylcarbonylamino)piperidin-4-ylethynyl]benzoate hydrochloride was synthesized according to Example 13, Steps 1-9 above.

### Step 10

Methyl 4-[4-(1H-indol-2-ylcarbonylamino)piperidin-4-ylethynyl]benzoate hydrochloride (207 mg, 0.47 mmol) in dichloromethane (5 mL) was treated with 2,6-lutidine (164 μL, 1.4 mmol), followed by (2,2,2-trifluoroethyl)phenyliodonium triflate (see Montanari, V.; Resnati, G. Tetrahedron Lett. 35, 8015, (1994)) (207 mg, 0.47 mmol). The reaction mixture was stirred at room temperature for 16 h and concentrated in vacuo. The crude methyl 4-[4-(1H-indol-2-ylcarbonylamino)-1-(2,2,2-trifluoroethyl)piperidin-4-ylethynyl]benzoate was used in the next step without further purification.

### Step 11

Methyl 4-[4-(1H-indol-2-ylcarbonylamino)-1-(2,2,2-trifluoroethyl)piperidin-4-yl-ethynyl]benzoate was converted to the title compound as described in Example 11, Step 5 above.

### BIOLOGICAL EXAMPLES

### Example 1

### Biomarkers Associated with the Antiproliferative Effect, In Vitro, of an HDAC Inhibitor

The antiproliferative effect of a compound of Formula (I) (Test Compound A) on HCT116 cell proliferation was determined. Proliferation was assayed in an Alamar Blue™ fluorometric assay as described by deFries and Mitsuhashi (1995). Briefly, HCT116 cells (5000 in 100 ul per well) were plated in 96-well plates in complete media (RPMI medium 1640 containing 10% (v/v) fetal bovine serum, 2 mM L-glutamine, 1 mM sodium pyruvate). Test Compound A was diluted from 20 mM stock solutions in DMSO. Serial dilutions were performed in medium containing 0.6% DMSO in wells (in triplicate) of a 96-well U-bottom plates starting with a 60 μM solution. After dilutions were completed, 100 μL of Test Compound A dilution (in triplicate) was transferred to designated triplicate wells of the 96-well plate containing cells in 100 μL of medium. Final concentrations of the dose-response for Test Compound A in assay plates ranged from 0.0015 to 10 μM. Control wells (cells with no treatment) received 100 μL of 0.6% DMSO in culture medium. The final DMSO concentration in each well was 0.3%. Wells containing medium with no cells served as the background wells. Cells were cultured with Test Compound A for 48 h.

Cell proliferation was assessed by measuring fluorescence after the addition of the fluorogenic redox indicator, Alamar Blue™ (BioSource International). Ten 1 L of Alamar Blue™ was added to each well of the 96-well plate(s) 4 hr prior to the end of the incubation period. Assay plates were read in a fluorescence plate reader (excitation, 530 nM; emission, 620 nM). The GI₅₀ value (concentration at which the growth of the tumor cells was inhibited by 50%) was determined by plotting the percent control fluorescence against the logarithm of Test Compound A concentration. Test Compound A inhibited HCT116 cell proliferation.

Next, the duration of exposure necessary to reach the GI₅₀ was determined. Briefly, HCT116 cells were plated in 96-well plates as described for the proliferation assay and pulsed with Test Compound A (0.3% final DMSO concentration) for varying lengths of time, washed and then incubated in drug-free media for the duration of the 48 h assay and the GI₅₀ values were calculated. Results demonstrated that up to 8 h of treatment had no effect on HCT116 cell proliferation (GI₅₀>10>M) while longer incubation times of 10-16 h did exhibit increasing antiproliferative effects. At 18 h the GI₅₀ value of 0.31 μM closely approached the 48 h GI₅₀ of 0.24 μM suggesting that 18 h exposure of HCT116 cells with Test Compound A was sufficient to commit the cells to the 48 h GI₅₀.

To understand the biochemical events associated with the antiproliferative effects of the Test Compound A, cellular levels of acetylated tubulin, phospho-H2AX and cytokeratin 18 fragment aa 387-397 were determined. Importantly, acetylated tubulin is a marker of HDAC inhibition while phospho-H2AX and cytokeratin 18 fragment aa 387-397 are early markers of apoptosis (Banath, J. and P. L. Olive (2003) Cancer Research 63:4347-4350; Biven, K. et al. (2003) Apoptosis 8:263-268. Carr, N. (2000) Arch. Pathol. Lab. Med. 124:1768-1772; de Fries, R. and M. Mitsuhashi (1995) JCI 9:89-95; Kramer, G. et al. (2004) Cancer Research 64:1751-1756; MacPhail, S. H. et al. (2003) Radiation Research 159:759-767; and Stiff, T. et al. (2004) Cancer Research 64:2390-2396.

For this purpose, HCT116 cells were pulsed for varying lengths of time (i.e., 5 mins, 15 mins, 1 h, 2 h, 6 h, 12 h and 18 h) with increasing concentrations of Test Compound A (0.01 μM, 0.1 μM, 0.5 μM, 5 μM and 10 μM; 0.2% final DMSO concentration) in 24-well plates. After treatment, the cells were collected and lysed in M-Per lysis buffer (Pierce) containing protease and phosphatase inhibitors as per the manufacturer's specifications. Lysates (20 μg total protein) were solubilized in SDS-PAGE reducing sample buffer, boiled and electrophoresed in 16% Tris-glycine gels (Invitrogen). The gels were then blotted onto nitrocellulose (22 um membrane; Invitrogen) and probed with either a monoclonal anti-acetylated tubulin antibody (Clone 6-11B-1; Sigma) or a polyclonal anti-phospho-H2AX antibody (Catalog number 2577, Phospho-Histone H2AX, Ser 139 Antibody; Cell Signaling). The blots probed with anti-acetylated tubulin antibody were then incubated with an anti-mouse peroxidase-conjugated secondary antibody (Pierce) and the blots were developed for enhanced chemiluminescence with the SuperSignal West Femto Maximum Sensitivity Substrate (Pierce) as per the manufacturer's specifications. The blots probed with anti-phospho-H2AX antibody were then incubated with a peroxidase-conjugated anti-rabbit secondary antibody and the blots were developed for enhanced chemiluminescence with the SuperSignal West Pico Kit (Pierce) as per the manufacturer's specifications. For detection of cytokeratin 18 fragment aa 387-397, a M30 Apoptosense ELISA kit (Peviva, Sweden; distributed by Alexis Biochemicals) was used in which cell lysates (5 μg total protein) were evaluated as per the manufacturer's specifications.

Results demonstrated that acetylated tubulin accumulated at all time points while phospho-H2AX and cytokeratin-18 fragment aa 387-397 were not detectable until 12 hr and 18 h of drug exposure. Taken together, the data suggest that accumulation of acetylated tubulin is a biomarker of HDAC inhibition but does not track with the antiproliferative effects of Test Compound A. However, the accumulation of both phospho-H2AX and cytokeratin-18 fragment aa 387-397 at 12 h and 18 h tracks with the antiproliferative effects of Test Compound A and thus phospho-H2AX and cytokeratin-18 fragment aa 387-397 appear to be pharmacoefficacy markers of cellular HDAC inhibition by Test Compound A.

### Example 2

### Early Biomarker Associated with the Anti-Tumor Response, In Vitro, of an HDAC Inhibitor

To understand the early biochemical events associated with the antiproliferative effects of the Test Compound B, cellular levels of phospho-H2AX was determined. Importantly, phospho-H2AX is an early marker of apoptosis. The accumulation of γ-H2AX, as an early indication of anti-tumor response, after administration of a compound of Formula (I) (Test Compound B) to HCT116 and HeLaS3 cells was determined.

To better understand the accumulation of γ-H2AX at earlier timepoints, two cell lines, HCT-116 and HeLaS3 were treated with the HDAC inhibitor Test Compound B and monitored for γ-H2AX by both Western blotting and cellular immunofluorescence. Both HCT-116 and HeLaS3 cells were grown in complete medium (McCoy's with 10% FBS and 1×Pen/Strep for HCT116 and DME/Ham F12 1:1 mix with 10% FBS, 2 mM L-Glutamine and 1×Pen/Strep for HeLaS3) in a 24 well dish or 4-well chamberslide overnight (18 h) then treated with Test Compound B from a 20 mM stock solution in DMSO to reach a final concentration of either 0, 0.1, 1, 3, or 10 μM in the well. The cells were grown incubated with compound for either one hour or two hours, at which point the media was removed and the cells washed once with phosphate buffered saline (PBS). For Western blot analysis, lysates from the treated and untreated cells (20 μg total protein) were electrophoresed and blotted onto PVDF, and the blots were probed with a polyclonal anti-phospho-H2AX antibody (purchased from Cell Signaling) at 1:1000 dilution. The blots were then incubated with an anti-rabbit IgG HRP-coupled secondary antibody at 1:10,000 dilution and developed for enhanced chemiluminescence detection. For cellular immunofluorescence staining, treated cells were washed once with PBS and the fixed and permeabilized cells were stained with monoclonal anti-phospho-H2AX antibody (from Upstate) at 1:500 dilution. The slides were then incubated with anti-mouse IgG AF488 (from Molecular Probes) at 1:2000 and mounted with Profound Gold Anti-fade with DAPI for immunofluorescence imaging.

Results from all experiments demonstrated a consistent accumulation of γ-H2AX in either HCT116 or HeLaS3 cells treated with 1 or 3 μM of Test Compound B at both the one hour and two hour timepoints. These results demonstrate a role of H2AX phosphorylation in the anti-tumor response of HDAC inhibitors, and also show that γ-H2AX can be detected in cells treated with HDAC inhibitors one hour following incubation with compound, and so represents an early biomarker of the activity of HDAC inhibitors.

### PHARMACEUTICAL COMPOSITION EXAMPLES

The following are representative pharmaceutical formulations containing a compound of Formula (I) or (II).

### Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | mL |
| colorings | 0.5 | mg |
| distilled water | q.s. to 100 | mL |

### Injectable Formulation

The following ingredients are mixed to form an injectable formulation.

| Ingredient | Amount |
|---|---|
| compound of this invention | 1.2 g |
| lactate buffer solution, 0.1M | 10.0 mL |
| HCl (1 N) or NaOH (1 N) | q.s. to suitable pH |
| saline (optional) | q.s. to suitable osmolarity |
| water (distilled, sterile) | q.s. to 20 mL |

Compound (1.2 g) is combined with 0.1 M lactate buffer (10 mL) and gently mixed. Sonication can be applied for several minutes if necessary to achieve a solution. Appropriate amount of acid or base is added q.s. to suitable pH (preferable pH 4). A sufficient amount of water is then added q.s. to 20 mL.

### Suppository Formulation

A suppository of total weight 2.5 g is prepared by mixing the compound of the invention with Witepsol™ H-15 (triglycerides of saturated vegetable fatty acid; Riches-Nelson, Inc., New York), and has the following composition:

| | |
|---|---|
| compound of the invention | 500 mg |
| Witepsol ™ H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application are hereby incorporated by reference in their entirety for all purposes to the same extent as if each individual patent, patent application or publication were so individually denoted.

1. A method of determining the anti-tumor activity of an HDAC inhibitor comprising measuring the level of phosphorylation of the histone variant H2AX before administration of the HDAC inhibitor and after administration at a timepoint before apoptosis can be detected.

2. A method of determining an efficacious dose for treating a cancer patient of which method comprises administering to the cancer patient different amounts of an HDAC inhibitor and determining, at a timepoint before apoptosis can be detected, the dose of the HDAC inhibitor that causes an increase in phosphorylation of the histone variant H2AX.

3. The method of claim 2 comprising:
(i) determining the level of γ-H2AX in the cancer patient prior to the administration of an HDAC inhibitor;
(ii) administering to the cancer patient different amounts of an HDAC inhibitor;
(iii) determining the level of γ-H2AX after administration of the HDAC inhibitor at said different amounts and at a timepoint before apoptosis can be detected; and
(iv) determining the efficacious dose by determining the increase in the level of γ-H2AX.

4. The method of claim 3 wherein the HDAC inhibitor is a compound of Formula (I): 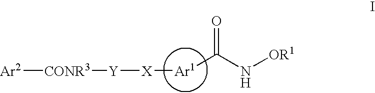
wherein:
R¹ is hydrogen or alkyl;
X is —O—, —NR²—, or —S(O)ₙ where n is 0-2 and R² is hydrogen or alkyl;
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
Ar¹ is phenylene or heteroarylene wherein said Ar¹ is optionally substituted with one or two groups independently selected from alkyl, halo, hydroxy, alkoxy, haloalkoxy, or haloalkyl;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl; and
individual stereoisomers, individual geometric isomers, or mixtures thereof; or a pharmaceutically acceptable salt thereof; or
a compound of Formula (II): 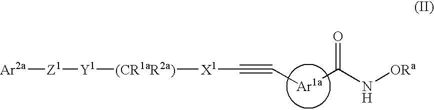
wherein:
R^{a} is hydrogen, alkyl, or alkylcarbonyl;
Ar^{1a} is arylene or heteroarylene wherein said Ar¹ is optionally substituted with one or two substituents independently selected from alkyl, halo, alkoxy, haloalkoxy, or haloalkyl;
X¹ and Y¹ are independently selected from bond or alkylene wherein alkylene is optionally substituted with halo, haloalkyl, hydroxy, alkoxy, haloalkoxy, amino, alkylamino, or dialkylamino;
R^{1a} is hydrogen or alkyl;
R^{2a} is hydrogen, alkyl, halo, haloalkyl, heteroalkyl, substituted heteroalkyl, aryl, heteroaryl, aralkyl, heteroaralkyl, hydroxyalkyl, alkoxyalkyl, or aminoalkyl; or
R^{1a} and R^{2a} together with the carbon to which they are attached form cycloalkylene or heterocycloalkylene;
Z¹ is —CONR^{3a}—, —NR⁴CO—, —SO₂NR⁵—, —NR⁶SO₂—, —NR⁷CONR⁸—, —NR⁹SO₂NR¹⁰—, —OCONR¹¹—, or —NR¹²COO— where R³-R¹² are independently selected from hydrogen, alkyl, hydroxyalkyl, haloalkyl, haloalkoxy, alkoxyalkyl, aralkyl, or heteroaralkyl; and
Ar^{2a} is aryl, aralkyl, aralkenyl, heteroaryl, heteroaralkyl, heteroaralkenyl, heterocycloalkyl, or heterocycloalkylalkyl; and
individual stereoisomers, individual geometric isomers, or mixtures thereof; or a pharmaceutically acceptable salt thereof provided that the hydroxamic acid and the acetylenic groups are not ortho to each other.

5. The method of claim 4 wherein the measurement after administration of the compound of Formula (I) or (II) is made 0 to 10 hours after said administration.

6. The method of claim 5 wherein the measurement after administration of the compound of Formula (I) or (II) is made 5 minutes to 8 hours after said administration.

7. (canceled)

8. The method of claim 4 wherein the compound of Formula (I) is 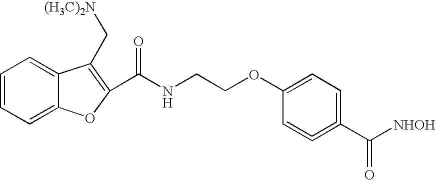
or a pharmaceutically acceptable salt thereof.

9. (canceled)

10. (canceled)

11. (canceled)

12. The method of claim 4 wherein the compound of Formula (II) is: 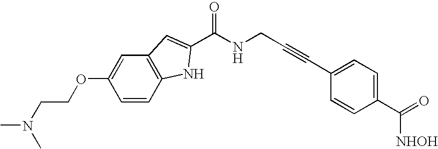
or a pharmaceutically acceptable salt thereof.

13. (canceled)

14. (canceled)

15. (canceled)

16. (canceled)

17. The method of claim 3 wherein the level of γ-H2AX is detected in vitro using anti-γ-H2AX antibody.

18. The method of claim 3 wherein the level of γ-H2AX is measured using blood or cancer tissue sample from the patient.

19. The method of claim 3 wherein the level of anti-γ-H2AX antibody complex is measured using immunofluorescence.

20. The method of claim 3 wherein the level of anti-γ-H2AX antibody complex is measured using immunoblotting methodology.

21. (canceled)

22. A method of determining an efficacious dose of a compound of Formula (I): 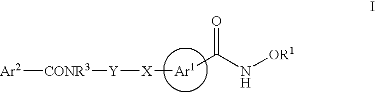
wherein:
R¹ is hydrogen or alkyl,
X is —O—, —NO₂—, or —S(O)ₙ where n is 0-2 and R² is hydrogen or alkyl,
Y is alkylene optionally substituted with cycloalkyl, optionally substituted phenyl, alkylthio, alkylsulfinyl, alkysulfonyl, optionally substituted phenylalkylthio, optionally substituted phenylalkylsulfonyl, hydroxy, or optionally substituted phenoxy;
Ar¹ is phenylene or heteroarylene wherein said Ar¹ is optionally substituted with one or two groups independently selected from alkyl, halo, hydroxy, alkoxy, haloalkoxy, or haloalkyl;
R³ is hydrogen, alkyl, hydroxyalkyl, or optionally substituted phenyl; and
Ar² is aryl, aralkyl, aralkenyl, heteroaryl, heteroaralkyl, heteroaralkenyl, cycloalkyl, cycloalkylalkyl, heterocycloalkyl, or heterocycloalkylalkyl; and
individual stereoisomers, individual geometric isomers, or mixtures thereof; or a pharmaceutically acceptable salt thereof;
for treating a cancer patient which method comprises administering to the cancer patient different amounts of a compound of Formula (I) and determining the dose of the compound of Formula (I) that causes an increase in phosphorylation of the histone variant H2AX and/or formation of cytokeratin-18 fragment aa 387-397.

23. The method of claim 22 comprising:
(i) determining the level of γ-H2AX in the cancer patient prior to the administration of a compound of Formula (I);
(ii) administering to the cancer patient different amounts of the compound of Formula (I);
(iii) determining the level of γ-H2AX after administration of the compound of Formula (I) at said different amounts; and
(iv) determining the efficacious dose of the compound of Formula (I) by determining the increase in the level of γ-H2AX.

24. The method of claim 22 comprising:
(i) determining the level of cytokeratin-18 fragment aa 387-397 in the cancer patient prior to the administration of a compound of Formula (I);
(ii) administering to the cancer patient different amounts of the compound of Formula (I);
(iii) determining the level of cytokeratin-18 fragment aa 387-397 after administration of the compound of Formula (I) at said different amounts; and
(iv) determining the efficacious dose of the compound of Formula (I) by determining the increase in the level of cytokeratin-18 fragment aa 387-397.

25. The method of claim 22 wherein the compound of Formula (I) is: 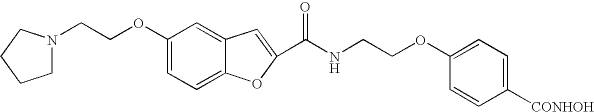
or a pharmaceutically acceptable salt thereof.

26. The method of claim 22 wherein the compound of Formula (I) is: 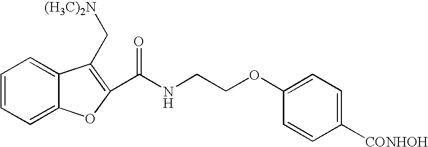
or a pharmaceutically acceptable salt thereof.

27. (canceled)

28. (canceled)

29. (canceled)

30. The method of claim 22 wherein the level of γ-H2AX and the level of cytokeratin-18 fragment aa 387-397 are detected in vitro using anti-γ-H2AX antibody and anti-cytokeratin-18 fragment antibody, respectively.

31. The method of claim 22 wherein the level of γ-H2AX is measured using blood or cancer tissue sample from the patient.

32. The method of claim 22 wherein the level of cytokeratin-18 fragment aa 387-397 is measured using serum sample or cancer tissue sample from the patient.

33. The method of claim 22 wherein the level of anti-γ-H2AX antibody complex and anti-cytokeratin-18 fragment aa 387-397 complex is measured using ELISA assay.

34. The method of claim 22 wherein the level of anti-γ-H2AX antibody complex and anti-cytokeratin-18 fragment aa 387-397 complex is measured using immunoblotting methodology.
